(19) United States
(12) Patent Application Publication
(10) Pub. No.: US 2026/0021140 A1
(43) Pub. Date: Jan. 22, 2026

(54) HBV Surface Antigen Specific T Cell Receptors And Uses Thereof

(71) Applicant: SCG CELL THERAPY PTE. LTD., Singapore (SG)

(72) Inventors: Ulrike PROTZER, Munich (DE); Karin WISSKIRCHEN, Munich (DE); Yanzhou HUANG, Shanghai (CN); Tao JIN, Shanghai (CN); Ke ZHANG, Shanghai (CN)

(21) Appl. No.: 18/871,858

(22) PCT Filed: Dec. 8, 2022
(86) PCT No.: PCT/SG2022/050892

§ 371 Date: Dec. 5, 2024

(30) Foreign Application Priority Data
Jun. 5, 2022 (CN) 202210626787.7

(51) Int. Cl. A61K 35/17; A61K 40/32; A61K 40/46; A61P 35/00; C07K 14/725; C07K 16/08

(52) U.S. Cl. CPC A61K 35/17; A61K 40/32; A61K 40/46; A61P 35/00; C07K 14/7051; C07K 16/082; A61K 2239/53; C07K 2317/565

(57) ABSTRACT

Provided are HLA-A02 restricted T cell receptors (TCRs) that specifically target HBV surface antigen which is a S20 peptide consisting of FLLTRILTI and the T cells expressing said TCRs. It also relates to the nucleic acid molecules and vectors encoding said TCRs and their medical uses in reducing HBV antigen-positive tumour cells and treating or preventing HBV infections and related diseases.

### CROSS-REFERENCE TO RELATED APPLICATIONS

This application is a national phase of PCT/SG2022/050892 filed on Dec. 8, 2022, which claims priority to Chinese Application No. 202210626787.7, which was filed on Jun. 5, 2022, the entire contents of both of which are incorporated herein by reference.

### FIELD OF THE INVENTION

The present invention relates to the technology field of immunotherapy, in particular to a T cell receptor targeting HBV surface antigen, a fragment thereof, a TCR polypeptide, a pharmaceutical composition and the use thereof.

### BACKGROUND OF THE INVENTION

### 1.1 Worldwide Prevalence of HBV

HBV is a non-cytopathic double-stranded DNA virus that is mainly transmitted through blood, mother-to-child transmission or sexual transmission. Although highly effective vaccines and anti-viral drugs are available nowadays, HBV still causes a significant disease burden in the worldwide.

In 2015, the global number of people living with HBV was 257 million, and the number of deaths caused by complications of chronic HBV infection exceeded 884,000. Estimation from epidemiological studies suggests that Asian and African countries have a higher proportion of HBV infection than Americas or Europe. In addition, due to the lack of correction function of HBV reverse transcriptase, HBV is prone to mutate and produce different genotypes. There are currently 10 different HBV genotypes distributed in different geographical regions.

### 1.2 the Most Common HBV Genotypes in Asians.

In Asian countries, the genotypes of HBV are mainly B and C subtypes. The researchers focused on the prevalence of different genotypes in different regions because of reported evidence shown that HBV genotypes affect clinical outcomes. For example, the progression of cirrhosis to HCC is much more likely to occur in genotype C and D than others.

### 1.3 HCC and its Oncogenic Pathways

HCC (Hepatocellular Carcinoma) is a primary hepatocellular carcinoma, which is the most common and fatal liver cancer in Asians. In 2017, HCC caused approximately 470,000 deaths due to long-term complications of chronic hepatitis infection. The molecular pathology of HCC is a complex process involving a variety of molecular aberrations and gene mutations, which ultimately leads to the occurrence of malignant diseases and the development of HCC.

Integration of HBV-DNA is seen in 80-90% of HBV-related HCCs. At the molecular level, the HBV interferes with the gene expression network of infected hepatocytes, and bring them into the carcinoma progression pathway. Integration of HBV-DNA increases genomic instability by affecting gene expression at insertion sites, thereby activating certain oncogenic pathways, such as phosphatidylinositide 3-kinase/Akt, myc, Wnt/β-catenin, c-Met and hedgehog et al. Previous studies have suggested that activation of Akt signaling transduction can inhibit transforming growth factor (TGF)-β-induced apoptosis and promote tumor formation and is also associated with β-catenin signaling transduction, thereby triggering hepatocellular carcinoma. Molecular changes caused by HBV-DNA integration also affect DNA damage checkpoints and lead to tumor formation in cirrhotic livers. These molecular alterations include loss of function of the p53 tumor suppressor gene, inactivation of the p27 cell cycle regulator, loss of heterozygosity at the insulin-like growth inhibitory 2 receptor site, and loss of expression of the p16 cell cycle arrestin protein.

At the same time, the viruses can also inhibit or block innate immunity, thereby affecting the development of an adaptive immune response. This chronic, persistent immune response manifests at the pathological level as hepatitis and long-term fibrosis, eventually leads to cirrhosis and liver cancer.

### 1.4 Current Status of HCC Treatment

Currently, surgical resection and liver transplantation are the most effective treatments for HCC, but less than 30% of HCC patients meet the criteria for these surgeries, and the postoperative recurrence rate of eligible patients is as high as 80% within 5 years. The waiting time for a donor liver is very long, the waiting list continues to rise, resulting in nearly 25 percent of patients not getting a transplant because of tumor progression.

The most commonly used conservative treatment is transcatheter arterial chemoembolization (TACE), but this therapy is contraindicated in advanced cirrhosis and hepatic decompensation. Because ischemic injury associated with embolism can lead to increased ascites and possibly death.

Currently, targeted drugs approved by FDA for the treatment of HCC are sorafenib and regafenib, both of which are tyrosine kinase inhibitors. However, clinical trial data in patients with advanced HCC suggests that these drugs may lead to an increased risk of bleeding and arteriovenous thrombosis. Moreover, the survival advantage in the indicated population is only 2-3 months, and disease progression is very common over time.

Chemotherapy generally does not provide a significant survival advantage for HCC patients, although several combinations with chemotherapy agents such as PIAF (cisplatin, interferon, doxorubicin, and 5-fluorouracil), GEMOX (gemcitabine and oxaliplatin), and FOLFOX4 (fluorouracil, calcium folinate, and oxaliplatin) are still under development. But clinical trials have not been successful and the results are incomplete.

In recent years, significant breakthroughs have been made in the immunotherapy of HCC. According to the IMbrave150, a Phase III first-line immunotherapy study, the results showed that the combination of atezolizumab and bevacizumab (T+A) significantly reduced the risk of death and disease progression compared with standard therapy of sorafenib monotherapy. However, the ORR (RECIST v1.1) was only 27.3%, and the mPFS was only 6.8 months. Among Chinese patients with a worse prognosis in advanced HCC, the mPFS was only 5.7 months. The above data indicates that the immunotherapy of HCC has made great progress compared with the past, but the efficacy is still unsatisfactory, and the immunotherapy of HCC needs to be further studied.

Patients with HBV-associated HCC need antiviral therapy to inhibit viral replication, reduce serum viral load, alleviate intrahepatic inflammatory response, and improve the prognosis related to cirrhosis. It is worth noting that although anti-viral therapy can effectively inhibit HBV replication and reduce hepatitis symptoms, it cannot clean the virus and control the virus after the treatment is stopped. Nucleosides and nucleoside analogs and interferons are often used in the clinical practice of anti-viral therapy for the prevention and treatment of HBV-related HCC, but the actual therapeutic efficacy of these products is uncertain and the clinical outcomes in terms of overall survival and disease recurrence remain controversial. Furthermore, long-term use of anti-viral drugs is associated with the development of drug resistance, and continued use can render these drugs ineffective. Prophylactic antiviral therapy with entecavir or tenofovir is recommended in patients treated with chemotherapy because of the potential for reactivation of HBV.

In conclusion, due to the limitations and high risks of the currently available treatment methods and drugs, new therapeutic strategies are needed to provide more options for patients with chronic hepatitis B and HBV-related HCC.

### 1.5 the Role of T Cells in the Immunotherapy of HBV-Associated HCC and the Rationale of Using TCR T Cells

T cells are immune cells derived from bone marrow, lymph and mature in the thymus. They express T cell antigen receptor (TCR) on their surface and play an important role in clearing infection and cancer cells in cell-mediated immunity. TCR recognizes and binds specifically to target epitopes presented by major histocompatibility complex (MHC) molecules. Once T cells recognize their target, they can kill the target cells by massive proliferation, cytokine release, and cytotoxicity.

Adoptive T cell immunotherapy has been tried to treat human malignancies such as leukemia and viral diseases such as cytomegalovirus (CMV) and Epstein-Barr virus (Epstein-Barr virus, EBV). However, isolation and expansion of viral or tumor-specific T cells from a patient's blood is difficult and time-consuming. Therefore, a novel therapeutic strategy was adopted to enable the gene-edited T lymphocytes to act on specific viral or tumor antigens by introducing T cell receptors (TCR) or TCRα/β heterodimers that target specific antigens, giving T cells with high antigen-specificity.

Hepatitis B virus synthesizes HBsAg protein in infected hepatocytes and assembles in the endoplasmic reticulum to form virus (sub-virus) particles that are secreted or arrive at the cell surface via physiological exchange of membranes. HBsAg expression is also commonly found in HBV-DNA integrated HCC cells.

Clinical evidence suggests that adoptive immunotherapy of HBV-specific T cells can control HBV replication or tumor growth. Leukemia patients can even acquire immunity to HBV after bone marrow transplantation by receiving bone marrow from donors who are specifically immune to HBV (either from HBV vaccination or from autoimmunity to achieve recovery from HBV infection). Similarly, transplantation of HBV-positive livers in subjects with specific immunity to HBV can also clearHBV infection in the transplanted livers.

Clinical evidence suggests that adoptive immunotherapy with HBV-specific T cells can control HBV replication and tumor growth. Patients with leukemia can even acquire immunity to HBV after bone marrow transplantation by receiving marrow from HBV-specific donors who have been vaccinated against HBV or rely on autoimmunity to achieve recovery from HBV infection. Similarly, transplantation of HBV-positive livers in subjects with specific immunity to HBV can also clear HBV infection in the transplanted livers.

However, HBV-specific T-cell immunity is severely inhibited in many patients with chronic hepatitis B and HBV-related HCC. In these patients, HBV-specific T cells are functionally deficient, scarce and prone to exhaustion. Due to these functional defects, HBV specific T cells are rarely detected by in vitro analysis of the patient's blood.

Therefore, constructing high-affinity TCR-T cells for reinfusion therapy have become a new option. Which includes using genetic engineering and other biological methods to develop specific TCRs targeting HBV surface antigens, isolating patient T cells and transfecting them in vitro. However, at present, there is no proven safe and effective TCR-T for the treatment of diseases caused by HBV infection, especially HBV-induced liver cancer.

### SUMMARY OF THE INVENTION

Through extensive and in-depth research, the application provides T cell receptors (TCRs) and modified TCR-T cells that specifically target HBV surface antigen, resulting in the elimination of liver cancer cells caused by HBV infection.

The application provides a novel TCR molecule or fragment thereof that specifically binds to the hepatitis B surface antigen FLLTRILTI-HLA-A2 complex. The T cell receptor (TCR) or fragment thereof, comprises:
a TCRα chain variable region comprising a αCDR3 having the amino acid sequence:
αCDR3: ATDERDDMR (SEQ ID NO:3), or a variant thereof in which one or two amino acids are replaced with another amino acid;
and;
a TCRβ chain variable region comprising a βCDR3 having the amino acid sequence:
βCDR3: ASSLNTEAF (SEQ ID NO:6) or a variant thereof in which one or two amino acids are replaced with another amino acid.

In other embodiments, the TCRα chain variable region comprising a αCDR3 having the amino acid sequence:
αCDR3: GADTSTDKLI (SEQ ID NO: 15), or a variant thereof in which one or two amino acids are replaced with another amino acid;
and;
the TCRβ chain variable region comprises a βCDR3 having the amino acid sequence:
βCDR3: ASSHGGAYEQY (SEQ ID NO:18) or a variant thereof in which one or two amino acids are replaced with another amino acid.

In other embodiments, The TCRα chain variable region comprising a αCDR3 having the amino acid sequence:
αCDR3: ATDAYGQNFV (SEQ ID NO: 24), or a variant thereof in which one or two amino acids are replaced with another amino acid;
and;
the TCRβ chain variable region comprising a βCDR3 having the amino acid sequence:
βCDR3: ASGSNTEAF (SEQ ID NO: 25) or a variant thereof in which one or two amino acids are replaced with another amino acid.

In some embodiments, The TCRα chain variable region comprising a αCDR1, a αCDR2 and a αCDR3 as shown in SEQ ID NO: 1, SEQ ID NO: 2 and SEQ ID NO:3, respectively;
and;
a TCRβ chain variable region comprising a βCDR1, a βCDR2 and a βCDR3 as shown in SEQ ID NO: 4, SEQ ID NO: 5 and SEQ ID NO:6, respectively;
or a variant thereof in which one or two amino acids in one or more of the CDRs are replaced with another amino acid.

In some embodiments, The TCRα chain variable region comprising a αCDR1, a αCDR2 and a αCDR3 as shown in SEQ ID NO: 13, SEQ ID NO: 14 and SEQ ID NO:15, respectively;
and;
the TCRβ chain variable region comprising a βCDR1, a βCDR2 and a βCDR3 as shown in SEQ ID NO: 16, SEQ ID NO: 17 and SEQ ID NO:18, respectively;
or a variant thereof in which one or two amino acids in one or more of the CDRs are replaced with another amino acid.

In some embodiments, the TCRα chain variable region comprising a αCDR1, a αCDR2 and a αCDR3 as shown in SEQ ID NO: 1, SEQ ID NO: 2 and SEQ ID NO:24, respectively;
and;
the TCRβ chain variable region comprising a βCDR1, a βCDR2 and a βCDR3 as shown in SEQ ID NO: 4, SEQ ID NO: 5 and SEQ ID NO:25, respectively;
or a variant thereof in which one or two amino acids in one or more of the CDRs are replaced with another amino acid.

In some embodiments, the TCRα chain variable region comprises the amino acid sequence that is at least 90% identical to any one of SEQ ID NO: 7, SEQ ID NO: 19 and SEQ ID NO: 26.

In some embodiments, the TCRβ chain variable region comprises the amino acid sequence that is at least 90% identical to any one of SEQ ID NO: 8, SEQ ID NO: 20 and SEQ ID NO: 27.

In some embodiments, the TCRα chain variable region comprises the amino acid sequence that is at least 90% identical to SEQ ID NO: 7. In some embodiments, the TCRβ chain variable region comprises the amino acid sequence that is at least 90% identical to SEQ ID NO: 8.

In some embodiments, the TCRα chain variable region comprises the amino acid sequence that is at least 90% identical to SEQ ID NO: 19. In some embodiments, the TCRβ chain variable region comprises the amino acid sequence that is at least 90% identical to SEQ ID NO: 20.

In some embodiments, the TCRα chain variable region comprises the amino acid sequence that is at least 90% identical to SEQ ID NO: 26. In some embodiments, the TCRβ chain variable region comprises the amino acid sequence that is at least 90% identical to SEQ ID NO: 27.

In some embodiments, the TCRα chain variable region comprises the amino acid sequence set forth in SEQ ID NO: 7, and the TCRβ chain variable region comprises the amino acid sequence set forth in SEQ ID NO: 8. In some embodiments, the TCRα chain variable region comprises the amino acid sequence set forth in SEQ ID NO:19, and the TCRβ chain variable region comprises the amino acid sequence set forth in SEQ ID NO: 20. In some embodiments, the TCRα chain variable region comprises the amino acid sequence set forth in SEQ ID NO: 26, and the TCRβ chain variable region comprises the amino acid sequence set forth in SEQ ID NO: 27.

In some embodiments, the TCR is an a heterodimer comprising a TCRα chain constant region TRAC*01 and a TCRβ chain constant region TRBC1*01 or TRBC2*01. The constant region could be selected from human TCR constant region sequences, or from murine constant region sequences. In some embodiments, the stability of the heterodimer increased and the rate of mismatches with the endogenous strand reduced by the introduction of at least one additional artificial disulfide bond in the constant region.

In some embodiments, the TCRα chain amino acid sequence set forth in SEQ ID NO: 10. In some embodiments, the TCRβ chain amino acid sequence set forth in SEQ ID NO: 11. In some embodiments, the TCRα chain amino acid sequence set forth in SEQ ID NO: 21. In some embodiments, the TCRβ chain amino acid sequence set forth in SEQ ID NO: 22. In some embodiments, the TCRα chain amino acid sequence set forth in SEQ ID NO: 28. In some embodiments, the TCRβ chain amino acid sequence set forth in SEQ ID NO: 29.

In some embodiments, the TCR is a single-chain fusion protein. In some embodiments, the single-chain TCR is formed by linking the TCRβ chain and the TCRα chain via P2A.

In some embodiments, from N-terminal to C-terminal the TCR comprises: T cell receptor beta chain variable region (TRBV), T cell receptor beta chain constant region (TRBC), P2A peptide, T cell receptor alpha chain variable region (TRAV) and T cell receptor alpha chain constant region (TRAC). In some embodiments, the amino acid sequence of the P2A peptide comprises the amino acid sequence set forth in SEQ ID NO: 9. In some embodiments, the amino acid sequence of the single-chain TCR set forth in SEQ ID NO: 12. In some embodiments, the amino acid sequence of the single-chain TCR set forth in SEQ ID NO: 23. In other embodiments, the amino acid sequence of the single-chain TCR set forth in SEQ ID NO: 30.

In some embodiments, cysteine residues form at least one artificial disulfide bond between the alpha and beta chain constant regions of the TCR.

In some embodiments, the TCR is soluble.

In some embodiments, the TCR comprises (a) all or a portion of a TCRα chain excluding the transmembrane region; and (b) all or a portion of a TCRβ chain excluding the transmembrane region; and both (a) and (b) comprise a functional variable region, or comprise a functional variable region and at least a portion of the TCR chain constant region.

In some embodiments, The TCR or fragment, which is capable of binding to a polypeptide of HBV surface antigen presented by HLA-A2. In some embodiments, the polypeptide comprises or consists of the amino acid sequence FLLTRILTI (SEQ ID NO: 31) or FLLTKILTI (SEQ ID NO:32).

In some embodiments, at least one conjugate is bound to the C- or N-terminus of the alpha chain and/or beta chain of the TCR, and the conjugate bound to the T cell receptor could be a detectable label, a therapeutic agent, PK-modifying moieties, or a combination of any of these.

The present application further provides a nucleic acid molecule encoding the TCR or fragment described above.

In some embodiments, the expression of TCR gene could be successfully improved by using codon optimization.

In some embodiments, the coding sequence of TRAV set forth in SEQ ID NO: 33. In some embodiments, the coding sequence of TRBV set forth in SEQ ID NO: 34. In some embodiments, the coding sequence of TRAV set forth in SEQ ID NO: 38. In some embodiments, the coding sequence of TRBV set forth in SEQ ID NO: 39. In some embodiments, the coding sequence of TRAV set forth in SEQ ID NO: 43. In some embodiments, the coding sequence f of TRBV set forth in SEQ ID NO: 44.

In some embodiments, the coding sequence of the TCRα chain set forth in SEQ ID NO: 35. In some embodiments, the coding sequence of the TCR beta chain set forth in SEQ ID NO: 36. In some embodiments, the coding sequence of the TCRα chain set forth in SEQ ID NO: 40. In some embodiments, the coding sequence of the TCR beta chain set forth in SEQ ID NO: 41. In some embodiments, the coding sequence of the TCRα chain set forth in SEQ ID NO: 45. In some embodiments, the coding sequence of the TCR beta chain set forth in SEQ ID NO: 46.

In some embodiments, the coding sequence of the TCR is a single chain, and the coding sequence of the TCRβ chain and TCRα chain are linked by the P2A coding sequence. In some embodiments, the single-stranded coding sequence of the TCR set forth in SEQ ID NO:37, SEQ ID NO:42, or SEQ ID NO:47.

The present application further provides an expression vector comprising the nucleic acid molecule as described above.

In some embodiments, the vector is selected from the group of plasmids, binary vectors, DNA vectors, mRNA vectors, retroviral vectors, lentiviral vectors, transposon-based vectors, and artificial chromosomes. In some embodiments, the vector is a viral vector; in some embodiments, the vector is a lentiviral vector.

The present application further provides polypeptides encoded by the nucleic acid molecule or the vector as described above.

The present application further provides a host cell, wherein the cell comprises the TCR or fragment, the nucleic acid molecule, the vector, or the polypeptide as described above.

In some embodiments, the cells are stem cells; in some embodiments, the cells are NK cells; in some embodiments, the cells are T cells; in some embodiments, the cells are CD4⁺ T cells, in some embodiments, the cell are CD8⁺ T cells.

The present application further provides a pharmaceutical composition which comprises the TCR or fragment, the nucleic acid molecule, the vector, the polypeptide, or the host cell and a pharmaceutically acceptable excipient, diluent or carrier.

The present application further provides the uses of the TCR or fragment, the nucleic acid molecule, the vector, the polypeptide, the host cells, or the pharmaceutical composition in preparation of medicines for preventing or treating HBV infection and other related diseases. In some embodiments, the diseases caused by HBV infection are hepatitis, liver fibrosis, liver cirrhosis, or liver cancer.

The present application further provides the TCR or fragment, the nucleic acid molecule, the vector, the polypeptide, the cell, or the pharmaceutical composition for use in treating HBV infection or diseases caused by HBV infection. In some embodiments, the diseases caused by HBV infection comprising one or more of hepatitis, liver fibrosis, liver cirrhosis, and liver cancer.

It should be understood that, within the scope of the present application, the above-mentioned technical features of the present application and the technical features specified in the following (e.g., the embodiments) can be combined with each other to constitute new or preferred technical solutions. Due to space limitations, all of them cannot be mentioned here.

### Beneficial Effects of this Application

The present application provides a new specific TCR or fragment targeting the HBV surface antigen. It is surprising to find that the TCR of the present application can target and recognize four genotypes of HBV surface antigen S20-28, including A, B, C and D genotypes. The TCR can recognize the most common HLA allele subtype in the HBV prevalent population. Therefore, there is a wider range of applicable populations for the TCR of the present application. The TCR-T cells of the present application have extremely strong in vitro and in vivo anti-viral abilities, and can specifically target and kill HBV-infected cells. Hence, the TCR could be used for the treatment of HBV infection and related diseases including hepatitis, liver fibrosis, liver cirrhosis, and liver cancer without off-target side-effects.

The current application, on the other hand, modifies the constant region by introducing an interchain disulfide bond via cysteines, improving the stability of the transduced TCR/heterodimer and reducing mispairing with endogenous TCR chains. In addition, the present application optimizes the expression codons of TCR to improve the expression efficiency.

### BRIEF DESCRIPTION OF THE DRAWINGS

FIG. 1: schematic diagram of the structure of a single-chain HBV S20 TCR.

FIG. 2: schematic diagram of the expression structure of HBV S20 TCR-T and its mechanism of action.

FIG. 3: titers of A1/B01/C01 TCR lentivirus.

FIG. 4: MOI detection of activated T cells infected with A01/B01/C01 TCR lentivirus.

FIG. 5: TCR expression level of A01/B01/C01 TCR-T.

FIG. 6: the S20-HLA02 expression level of HepG2-LMS-LG cells.

FIG. 7: cytotoxicity of A01 TCR-T on HepG2-LMS-LG cells with different ratios of effector to target (E:T) cells.

FIG. 8: cytotoxicity and cytokine profiles of B01 TCR-T on HepG2-LMS-LG cells with different ratios of effector to target (E:T) cells.

FIG. 9: cytotoxicity and cytokine profiles of C01 TCR-T on HepG2-LMS-LG cells with different ratios of effector to target (E:T) cells.

FIG. 10: cytotoxicity comparison of A01/B01/C01 TCR-T on HepG2-LMS-LG cells.

FIG. 11: functionalities of different HBV S20 TCR-T subsets on target cells.

FIG. 12: identification of the key amino acids of S20 polypeptide recognized by A01/B01/C01 TCR-T.

FIG. 13: cross-reactivity determination of A01/B01/C01 TCR-T to human peptide database.

FIG. 14: binding capacity determination of A01/B01/C01 TCR-T to S20 of different genotypes.

FIG. 15: A01/B01/C01 TCR-T identifies different subtypes of HLA-A02.

FIG. 16A-C: anti-tumor activities of A01 TCR-T in HepG2-LMS-LG CDX xenograft model.

FIG. 17A-B: anti-tumor activities of B01 TCR-T in HepG2-LMS-LG CDX xenograft model.

FIG. 18A-C: anti-tumor activities of C01 TCR-T in HepG2-LMS-LG CDX xenograft model.

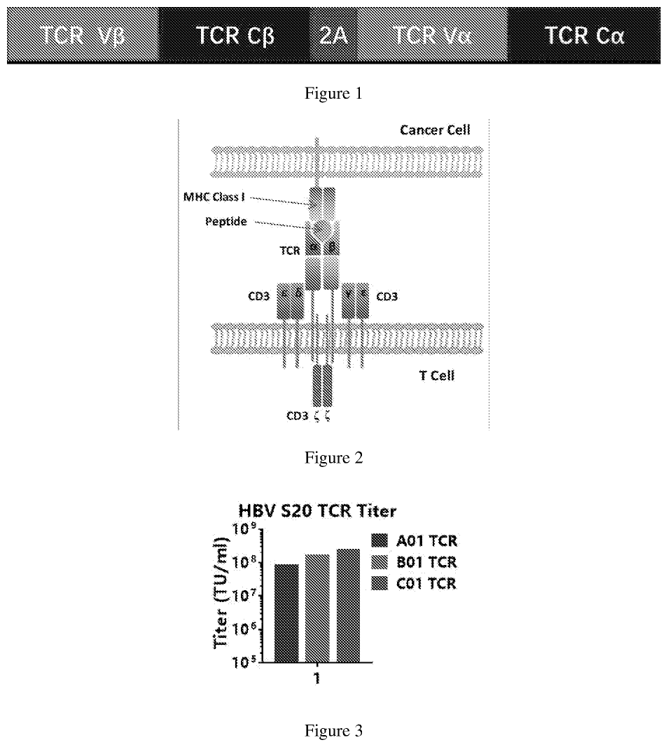

### DETAILED DESCRIPTION OF THE INVENTION

### Definitions

In order to more readily understand the invention, certain technical and scientific terms are specifically defined below. Unless specifically defined elsewhere in this document, all other technical and scientific terms used herein have the meaning commonly understood by one of ordinary skill in the art to which this invention belongs.

As used herein, “hepatitis B surface antigen T cell receptor (TCR)” refers to a TCR that binds to a complex of the major histocompatibility complex (MHC) and HBV surface antigen to induce helper or cytotoxic responses. Specifically, the HBV surface antigen could be HBs20-28, which can be used interchangeably with HBs20, HBs20-28, S20-28 and S20 in this application. Unless otherwise specified, it refers to the S20-28 antigen of genotypes A and D with the amino acid sequence FLLTRILTI. In some embodiments, the HBV surface antigens are HBV genotypes A and D; in some embodiments, the HBV surface antigens are HBV genotypes B and C. In some embodiments, the HBV surface antigen comprises the FLLTRILTI (SEQ ID NO: 31) amino acid sequence; in some embodiments, the HBV surface antigen comprises the FLLTKILTI (SEQ ID NO: 32) amino acid sequence. In some embodiments, the HBV surface antigen has the amino acid sequence of FLLTRILTI (SEQ ID NO: 31); in some embodiments, the HBV surface antigen has the amino acid sequence of FLLTKILTI (SEQ ID NO: 32).

The term “MHC molecule” refers to a protein of the immunoglobulin superfamily, which may be class I or class II MHC molecules. It is specific for antigen presentation, and different individuals have different MHCs, which can present different short peptides in a protein antigen to the APC cell surfaces. The human MHC is often referred to as the HLA gene or HLA complex.

TCR is a glycoprotein found on the surface of the T cell membrane that exists as α-chain/β-chain or γ-chain/δ-chain heterodimers.δ TCR heterodimers consist of alpha and beta chains in 95% of T cells, while 5% of T cells have TCRs composed of gamma and delta chains. A native heterodimeric TCR has an α chain and a β chain, and the α chain and the β chain chain are the heterodimeric TCR's subunits. In a broad sense, alpha and beta chains contain variable region, linker, and constant region, respectively. The beta chain typically also comprises a short variable region between the variable region and linker region, but the short variable region is often considered as a part of the linker region. The variable region includes 3 CDRs (complementarity determining regions), CDR1, CDR2 and CDR3, respectively. The CDRs are chimerized in the framework regions. The CDR regions of the α chain and the β chain of the present application are delimited using the IMGT numbering system. The CDR regions determine the binding of TCR to the pMHC complex. The sequences of the TCR constant regions can be found in the public database of IMGT. For example, the constant region sequence of the alpha chain is “TRAC*01”, and the constant domain sequence of the beta chain is “TRBC1*01” or “TRBC2*01”.

In this application, the terms “T cell receptor”, “TCR”, “TCR molecule” are used interchangeably.

### TCR Molecule

The TCRs or fragments of the present application recognize HBV surface antigens that are HLA-A2 restricted. Approximately 50% of the general population expresses the MHC class I molecule HLA-A2, an HLA-A serotype. Therefore, HLA-A2-restricted TCRs may find widespread therapeutic use. In particular, the subtype may identify gene products of many HLA-A*02 alleles, comprising HLA-A*0201,*0202, *0203, *0206, and *0207 gene products. There may be distinct differences in the subtypes between Caucasian and Asian populations, whereas more than 95% of the HLA-A2 positive Caucasian population is HLA-A0201. The HLA-A2 positive Chinese population may be broken down into 23% HLA-A0201; 45% HLA-A0207; 8% HLA-A0206; 23% HLA-A0203.

In some embodiments, the TCR comprises a TCR alpha chain variable region and a TCR beta chain variable region having 3 complementarity determining regions (CDR), respectively.

In some embodiments, a TCRα chain variable region comprising a αCDR3 having the amino acid sequence: αCDR3: ATDERDDMR (SEQ ID NO: 3), or a variant thereof in which one or two amino acids are replaced with another amino acid;
and/or;
a TCRβ chain variable region comprising a βCDR3 having the amino acid sequence: βCDR3: ASSLNTEAF (SEQ ID NO: 6) or a variant thereof in which one or two amino acids are replaced with another amino acid.

In other embodiments, the TCRα chain variable region comprising a αCDR3 having the amino acid sequence: αCDR3: GADTSTDKLI (SEQ ID NO: 15), or a variant thereof in which one or two amino acids are replaced with another amino acid;
and/or;
the TCRβ chain variable region comprising a βCDR3 having the amino acid sequence: βCDR3: ASSHGGAYEQY (SEQ ID NO:18) or a variant thereof in which one or two amino acids are replaced with another amino acid.

In other embodiments, The TCRα chain variable region comprising a αCDR3 having the amino acid sequence: αCDR3: ATDAYGQNFV (SEQ ID NO: 24), or a variant thereof in which one or two amino acids are replaced with another amino acid;
and/or;
the TCRβ chain variable region comprising a βCDR3 having the amino acid sequence: βCDR3: ASGSNTEAF (SEQ ID NO:25) or a variant thereof in which one or two amino acids are replaced with another amino acid.

In some embodiments, The TCRα chain variable region comprising a αCDR1, a αCDR2 and a αCDR3 as shown in SEQ ID NO: 1, SEQ ID NO: 2 and SEQ ID NO:3, respectively;
and/or;
a TCRβ chain variable region comprising a βCDR1, a βCDR2 and a βCDR3 as shown in SEQ ID NO: 4, SEQ ID NO: 5 and SEQ ID NO: 6, respectively;
or a variant thereof in which one or two amino acids in one or more of the CDRs are replaced with another amino acid.

In some embodiments, The TCRα chain variable region comprising a αCDR1, a αCDR2 and a αCDR3 as shown in SEQ ID NO: 13, SEQ ID NO: 14 and SEQ ID NO: 15, respectively;
and/or;
the TCRβ chain variable region comprising a βCDR1, a βCDR2 and a βCDR3 as shown in SEQ ID NO: 16, SEQ ID NO: 17 and SEQ ID NO: 18, respectively;
or a variant thereof in which one or two amino acids in one or more of the CDRs are replaced with another amino acid.

In some embodiments, the TCRα chain variable region comprising a αCDR1, a αCDR2 and a αCDR3 as shown in SEQ ID NO: 1, SEQ ID NO: 2 and SEQ ID NO: 24, respectively;
and/or;
the TCRβ chain variable region comprising a βCDR1, a βCDR2 and a βCDR3 as shown in SEQ ID NO: 4, SEQ ID NO: 5 and SEQ ID NO: 25, respectively;
or a variant thereof in which one or two amino acids in one or more of the CDRs are replaced with another amino acid.

Chimeric TCRs can be prepared by inserting the described amino acid sequences of the CDR regions of the present application into any suitable framework structure, as long as the framework structure is compatible with the CDR regions. The skilled artisan can design or synthesize TCR molecules with corresponding functions based on the CDR regions disclosed in the present application. Therefore, the TCR in the present application refers to a TCR comprising the above-mentioned a and/or B chain CDR region amino acids sequences and any suitable framework structure. The amino acid sequence of the TCRα chain variable region of the present application having at least 90%, preferably 95%, more preferably 98% sequence identity to SEQ ID NO:7, SEQ ID NO: 19 or SEQ ID NO: 26; and/Or, the amino acid sequence of the TCRβ chain variable region having at least 90%, preferably 95%, more preferably 98% sequence identity to SEQ ID NO: 8, SEQ ID NO: 20 or SEQ ID NO: 27.

In some embodiments, the TCRα chain variable region comprises the amino acid sequence set forth in SEQ ID NO: 7. In some embodiments, the TCRβ chain variable region comprises the amino acid sequence set forth in SEQ ID NO: 8. In some embodiments, the TCRα chain variable region comprises the amino acid sequence set forth in SEQ ID NO: 19. In some embodiments, the TCRβ chain variable region comprises the amino acid sequence set forth in SEQ ID NO: 20. In some embodiments, the TCRα chain variable region comprises the amino acid sequence set forth in SEQ ID NO: 26.In some embodiments, the TCRβ chain variable region comprises the amino acid sequence set forth in SEQ ID NO: 27.

In some embodiments, the amino acid sequence of the variable region of the TCRα chain set forth in SEQ ID NO: 7, and the amino acid sequence of the variable region of the TCRβ chain set forth in SEQ ID NO: 8. In some embodiments, the amino acid sequence of the variable region of the TCRα chain set forth in SEQ ID NO: 19, and the amino acid sequence of the variable region of the TCRβ chain set forth in SEQ ID NO: 20. In some embodiments, the amino acid sequence of the variable region of the TCRα chain set forth in SEQ ID NO: 26, and the amino acid sequence of the variable region of the TCRβ chain set forth in SEQ ID NO: 27.

In some embodiments, the TCR is up heterodimer comprising a TCRα chain constant region and a TCRβ chain constant region. In some embodiments, the constant regions of the TCR molecules of the present application are human constant regions. Those skilled in the art know or can obtain the human constant region amino acid sequence by consulting relevant books or the public database of IMGT. For example, the constant region sequence of the alpha chain can be “TRAC*01”, and the constant region sequence of the beta chain can be “TRBC1*01” or “TRBC2*01”. In some embodiments, additional disulfide bonds are introduced into the constant region to improve stability and reduce mismatches between exogenous TCR molecules and endogenous TCR molecules. The constant region may also be mouse constant region. Replacing TRAC and TRBC with mouse-derived constant domains can avoid the mismatch between exogenous TCR molecules and endogenous TCR molecules. This effect is similar to the purpose of exogenous introduction of artificial disulfide bonds.

In some embodiments, the amino acid sequence of the alpha chain of the TCR set forth in SEQ ID NO:10, and/or the amino acid sequence of the beta chain of the TCR set forth in SEQ ID NO:11. In some embodiments, the amino acid sequence of the alpha chain of the TCR set forth in SEQ ID NO:21, and/or the amino acid sequence of the beta chain of the TCR set forth in SEQ ID NO:22. In some embodiments, the amino acid sequence of the alpha chain of the TCR set forth in SEQ ID NO:28, and/or the amino acid sequence of the beta chain of the TCR set forth in SEQ ID NO:29.

In some embodiments, the TCR molecules of the present application are single-chain consisting of part or all of an alpha chain, and/or, part or all of a beta chain. In some embodiments, the single-chain TCR is formed by linking the amino acid sequence of TCR β chain and the amino acid sequence of TCR α chain through P2A. In a preferred embodiment of the present application, the T cell antigen receptor polypeptide from N-terminal to C-terminal includes: TRBV, TRBC, P2A, TRAV and TRAC. In another preferred embodiment of the present application, the T cell antigen receptor polypeptide from N-terminal to C-terminal includes: TRAV, TRAC, P2A, TRBV and TRBC.

In some embodiments, the alpha chain variable region of the single-chain TCR molecule comprises CDR1 (SEQ ID NO: 1), CDR2 (SEQ ID NO: 2), and CDR3 (SEQ ID NO: 3); preferably, the alpha chain variable region as shown in SEQ ID NO: 7. The beta chain variable region of the single-chain TCR molecule comprises CDR1 (SEQ ID NO: 4), CDR2 (SEQ ID NO: 5) and CDR3 (SEQ ID NO: 6); preferably, the beta chain variable region as shown in SEQ ID NO: 8.

In some embodiments, the alpha chain variable region of the single-chain TCR molecule comprises CDR1 (SEQ ID NO: 13), CDR2 (SEQ ID NO: 14), and CDR3 (SEQ ID NO: 15); preferably, the alpha chain variable region as shown in SEQ ID NO: 19. The beta chain variable region of the single-chain TCR molecule comprises CDR1 (SEQ ID NO: 16), CDR2 (SEQ ID NO: 17) and CDR3 (SEQ ID NO: 18); preferably, the beta chain variable region as shown in SEQ ID NO: 20.

In some embodiments, the alpha chain variable region of the single-chain TCR molecule comprises CDR1 (SEQ ID NO: 1), CDR2 (SEQ ID NO: 2), and CDR3 (SEQ ID NO: 24); preferably, the alpha chain variable region as shown in SEQ ID NO: 26. The beta chain variable region of the single-chain TCR molecule comprises CDR1 (SEQ ID NO: 4), CDR2 (SEQ ID NO: 5) and CDR3 (SEQ ID NO: 25); preferably, the beta chain variable region as shown in SEQ ID NO: 27.

In some embodiments, the amino acid sequence of the single-chain TCR set forth in SEQ ID NO: 12. In some embodiments, the amino acid sequence of the single-chain TCR set forth in SEQ ID NO: 23. In some embodiments, the amino acid sequence of the single-chain TCR set forth in SEQ ID NO: 30.

The naturally TCR is a membrane protein that is stabilized by its transmembrane region. Like immunoglobulins (antibodies) as antigen recognition molecules, TCRs can also be developed for diagnostic and therapeutic applications, where soluble TCR molecules need to be obtained.

The soluble TCR molecules do not include their transmembrane domains. They have a wide range of uses, not only to study the interaction of TCR with pMHC, but also as a diagnostic tool to detect infection or as a marker for autoimmune diseases. The soluble TCRs can be used to deliver therapeutic agents (e.g., cytotoxic or immunostimulatory compounds) to cells presenting specific antigens. The soluble TCRs can also be conjugated to other molecules (e.g., anti-CD3 antibodies) to redirect T cells to target cells presenting specific antigens.

In some embodiments, the TCR is soluble.

In some embodiments, the TCR comprises (a) all or a portion of a TCRα chain excluding the transmembrane domain; and (b) all or a portion of a TCRβ chain excluding the transmembrane domain; and both (a) and (b) comprise a functional variable domain, or comprise a functional variable domain and at least a portion of the TCR chain constant domain.

In some embodiments, there are artificial disulfide bonds between the alpha and beta chain constant regions of the soluble TCR.

The soluble TCR may be prepared by any method known in the art. Examples of processes that may be used to prepare the soluble TCR may comprise but are not limited to constructing polymeric receptor chains in which an immunoglobulin heavy chain variable region from at least one phosphorylcholine-specific antibody may be substituted with TCR α and β variable regions, introducing translational termination codons upstream of the TCR transmembrane region or replacing the transmembrane domains of the TCR α and β chain cDNAs with a signal for glycosylphosphatidyl inositol (GPI) linkage from the carboxy terminus of the GPI linked protein Thy-1.

The soluble TCR of the present application can be used alone, or combined with the conjugate in a covalent or other manner, preferably in a covalent manner. The conjugates include a detectable label, a therapeutic agent, a PK-modifying moiety, or a combination or conjugation of any of these.

Detectable labels for diagnostic purposes include, but are not limited to, fluorescent or chemiluminescent labels, radioactive labels, MRI (magnetic resonance imaging) or contrast medium of CT (electronic computer X-ray tomography technique), or enzymes capable of producing detectable products.

Therapeutic agents that can be conjugated to the TCRs of the present application include, but are not limited to, chemotherapeutic agents (e.g., cisplatin), prodrug-activating enzymes, cytokines, toxins (e.g., PE38, calcimycin, or diphtheria toxin), immunomodulatory antibody fragments (e.g., anti-CD3 or anti-CD16, Fc fragments, scFv), radionuclides, viral particles, liposomes, gold nanoparticles, nanomagnetic particles or nanoparticles of any form, etc.

The soluble TCR may be linked to at least one anti-viral drug. The anti-viral drug may target HBV. For example, anti-viral drugs may comprise, but are not limited to, adefovir dipivoxil, interferon alfa-2b, pegylated interferon alfa-2a, lamivudine, entecavir, telbivudine and the like.

The TCRs of the present application may also be provided in the form of multivalent complexes. The multivalent TCR complex of the present application comprises two, three, four or more multimers formed by combining the TCRs of the present application, for example, the tetramerization domain of p53 can be used to generate tetramers, or a complex formed by binding of one or more TCRs of the present application to another molecule. Compared with the non-multimeric wild type or the T cell receptor heterodimer of the present application, the binding ability of the multivalent TCR complex of the present application to the FLLTRILTI-HLA-A*02 complex can be enhanced. Therefore, the multivalent complex of the TCR of the present application also belongs to the present application. The TCR complexes of the present application can be used to track or target cells presenting specific antigens in vitro or in vivo, as well as to generate intermediates for other multivalent TCR complexes with such applications.

### Nucleic Acid Molecule

The application provides nucleic acid molecules encoding the TCR molecules described herein above, or fragments thereof, which may be one or more CDRs, variable regions of alpha and/or beta chains, alpha and/or beta chains.

In some embodiments, the nucleic acid encodes one or more structural features for increasing and/or stabilizing the association between the expressed TCR alpha and beta chains. In some embodiments, the characteristic may be a specific amino acid or amino acid sequence. In some embodiments, the nucleic acid may encode one or more unnatural cysteine residues for forming one or more disulfide bonds between the TCR alpha and beta chains. In some embodiments, the nucleic acid may encode one or more non-native cysteine residues in the constant domains of the TCR alpha and beta chains.

The nucleic acid molecules of the present application may be single-stranded or double-stranded, the nucleic acid molecules may be RNA or DNA, and contain introns or not. Preferably, the nucleic acid molecules of the present application do not contain introns but are capable of encoding the TCRs of the present application or fragments thereof.

The expression of TCR gene could be successfully improved by codon optimization. Different biased codons are preferred in different species. Depending on the type of cell, the codons in the sequence can be changed to increase the amount of expression. Codon usage tables for mammalian cells, as well as various other organisms, are well known to those skilled in the art.

The nucleic acid sequence encoding the TRAV of the present application set forth in SEQ ID NO:33, SEQ ID NO:38 or SEQ ID NO:43. and/or the nucleic acid sequence encoding the TRBV set forth in SEQ ID NO:34, SEQ ID NO:39 or SEQ ID NO:44.

The nucleic acid sequence encoding the TCRα chain of the present application set forth in SEQ ID NO: 35, SEQ ID NO: 40 or SEQ ID NO: 45. and/or the nucleic acid sequence encoding the TCR beta chain set forth in SEQ ID NO: 36, SEQ ID NO: 41 or SEQ ID NO: 46.

In some embodiments, the coding sequence of the TCR is single-stranded, and the coding sequence of the TCRβ chain and the coding sequence of the TCRα chain are linked by the P2A coding sequence, and the single-stranded coding nucleic acid are in the same reading frame. In some embodiments, the single-stranded coding sequence of the TCR set forth in SEQ ID NO: 37, SEQ ID NO: 42 or SEQ ID NO: 47.

### Expression Vector

The present application provides at least one “vector” as a medium (DNA or RNA) for the transfer of exogenous nucleic acid into a cell. The vector may be an expression vector for expressing nucleic acid in the cell. Such vectors may contain a promoter sequence operably linked to the nucleic acid encoding the sequence to be expressed. The vector may also contain stop codons and expression enhancers.

In some embodiments, the present application provides at least one construct comprising the polynucleotide of the present application operably connected to at least one promoter. The coding sequences for a and β chains of the TCR may be operably connected to at least one promoter functional in the isolated cell. Suitable promoters may be constitutive and inducible promoters, and the selection of an appropriate promoter may be well within the skill in the art. For example, suitable promoters may comprise, but are not limited to, the retroviral LTR, the SV40 promoter, the CMV promoter and cellular promoters (e.g., the P-actin promoter).

In some embodiments, the present application provides at least one vector comprising the construct according to the present application or the polynucleotide according to the present invention. In particular, the vectors may comprise, but not limited to, plasmids, binary vectors, mRNA vectors, lentiviral vectors, retroviral vectors, adenoviral vectors, adeno-associated virus vectors and Herpes Simplex Virus vectors. More in particular, lentiviral vectors may be used for delivery of the constructs either in vitro, ex vivo or in vivo, as described in the examples.

### Cells

The present application also includes isolated cells expressing the TCRs and/or fragments, wherein the cells may be stem cells or immune cells. The immune cells can be T cells, natural killer cells, dendritic cells or macrophages. In some embodiments, the immune cells are T cells. The T cells can be derived from T cells isolated from a subject, or can be part of a mixed population of cells isolated from a subject, such as a peripheral blood lymphocyte (PBL) population. For example, the cells can be isolated from peripheral blood mononuclear cells (PBMCs), CD4⁺ helper T cells, or CD8⁺ cytotoxic T cells. The cells may be in a mixed population of CD4⁺ helper T cells/CD8⁺ cytotoxic T cells. Typically, the cells can be activated with antibodies (e.g., anti-CD3 antibodies) to render them more receptive to transfection, e.g., with a vector comprising a nucleic acid sequence encoding a TCR molecule of the present application. In some embodiments, the cells of the present application may also be stem cells, such as hematopoietic stem cells (HSCs). Transfer of the TCR gene to HSC does not result in expression of TCR on the cell surface because the CD3 molecule is not expressed on the surface of stem cells. However, when HSCs differentiate into lymphoid precursors that migrate to the thymus, the expression of CD3 molecules will initiate the expression of TCR molecules. Cells expressing the TCR or fragments of the present application may be suitable for use in adoptive transfer protocols to provide a particularly effective mode of treatment. The cells of the present application can overcome the problem of HBV-specific CD8⁺ and CD4⁺ cells which are absent or poorly functioning in patients.

There are numbers of methods suitable for transfection of T cells with DNA or RNA encoding the TCR of the present application or fragments thereof (e.g., Robbins et al. (2008) J. Immunol. 180:6116-6131). Methods of introducing polynucleotide molecules or vectors into cells are known in the art. The vectors can be readily introduced into host cells, e.g., mammalian, bacterial, yeast or insect cells, by any method known in the art. For example, the expression vector can be transferred into host cells by physical, chemical or biological means.

The physical methods of introducing polynucleotides into host cells include calcium phosphate precipitation, lipofection, particle bombardment, microinjection, electroporation, and the like. The biological methods for introducing polynucleotides of interest into host cells include the use of DNA and RNA vectors. The chemical means of introducing polynucleotides into host cells include colloidal dispersion systems, such as macromolecular complexes, nanocapsules, microspheres; and lipid-based systems, including oil-in-water emulsions, micelles, mixed micelles, and liposomes.

The present application also provides a method for producing a TCR, fragment or polypeptide according to the application, comprising introducing a vector from the present invention into cells and culturing the cell under conditions suitable for expression of the vector by the cell.

Any cell suitable for the expression of polypeptides may be used for producing TCRs, fragments and polypeptides according to the invention. The cell may be a prokaryote or eukaryote. Suitable prokaryotic cells include E. coli. Examples of eukaryotic cells include a yeast cell, a plant cell, insect cell or a mammalian cell. In some cases the cell is not a prokaryotic cell because some prokaryotic cells do not allow for the same post-translational modifications as eukaryotes. In addition, very high expression levels are achievable in eukaryotes and proteins can be easily purified from eukaryotes using appropriate tags. Specific plasmids may also be utilized which enhance secretion of the TCR, fragment or polypeptide into the media.

### Compositions

The present application also provides compositions comprising a TCR, fragment, nucleic acid, vector, polypeptide or cell according to the present invention. In some embodiments, the composition is a pharmaceutical composition. In some embodiments, the composition is a composition suitable for use in research, therapy, prophylaxis and/or diagnosis.

In some embodiments, the TCR, fragment, nucleic acid, vector, polypeptide or cell according to the present invention preferably formulated as a medicament or pharmaceutical together with one or more other pharmaceutically acceptable ingredients well known to those skilled in the art, including, but not limited to, pharmaceutically acceptable carriers, adjuvants, excipients, diluents, fillers, buffers, preservatives, anti-oxidants, lubricants, stabilizers, solubilizers, surfactants (e.g., wetting agents), masking agents, colouring agents, flavouring agents, and sweetening agents. The term “pharmaceutically acceptable” as used herein pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, adjuvant, excipient, etc. must also be “acceptable” in the sense of being compatible with the other ingredients of the formulation. Suitable carriers, adjuvants, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 2nd edition, 1994.

### Medicinal Use

In another aspect, use of the TCR or fragments, nucleic acid, vector, polypeptide, cells or pharmaceutical composition of the present application in the preparation of a medicament for the treatment or prevention of a disease or disorder is provided.

In some embodiments, the TCR or fragments, nucleic acids, vectors, polypeptide, cells or pharmaceutical composition of the present application can be used to prevent or treat diseases caused by HBV infection. The diseases caused by HBV infection include acute hepatitis (including fulminant liver failure), chronic hepatitis, liver fibrosis, liver cirrhosis, liver cancer such as hepatocellular carcinoma (HCC), or pancreatic cancer.

### Methods of Treatment and Prophylaxis

Treatment or prevention can be performed by isolating T cells from patients or volunteers suffering from diseases caused by HBV infection. Introducing the TCR of the present application into the above T cells, and then these genetically engineered cells are infused back into the patient. Therefore, the present application provides a method for the treatment of diseases caused by HBV infection by infusing the isolated T cells expressing the TCR of the present application into the patients. Preferably, the T cells are derived from the patient. Generally, it includes (1) isolating T cells from a patient, (2) transducing T cells in vitro with the nucleic acid molecules or vectors capable of encoding the TCR molecules of the present application, and (3) infusing the genetically engineered T cells into the patient in vivo. The number of cells isolated, transfected, and reinfused can be determined by the physician.

The technical solutions of the present application will be clearly and completely described below with reference to specific embodiments. Obviously, the described embodiments are only a part of the embodiments of the present application, rather than all the embodiments. Based on the embodiments in the present application, all other embodiments obtained by those skilled in the art without creative work fall into the protection scope of the present application. It should be noted that the description order of the following embodiments is not intended to limit the preferred order of the embodiments.

In the following examples, the experimental methods of unrecited specific conditions are usually in accordance with conventional conditions such as those in Molecular cloning by Sambrook et al.: Laboratory Manual (Molecular cloning-A Laboratory manual), or according to the methods proposed by manufacturer. The percentages and parts are by weight unless otherwise indicated. Unless otherwise specified, the reagents and materials involved in the text can be obtained commercially, or prepared by those skilled in the art according to common knowledge. Any methods and materials similar or equivalent to those described can be used in this application. The preferred implementation methods and materials herein are for exemplary purposes only, but do not limit the content of this application.

### Example 1: Acquisition of Target Gene of HBV S20 TCR and Construction of its Vector

### Acquisition of Target Gene of HBV S20 TCR:

Peripheral blood mononuclear cells (PBMCs) were isolated from fresh blood of HBV-infected recovered volunteers (HLA-A2+). 1 nM S20 polypeptide (FLLTRILTI) and T2 cells (American typical Species collection) were incubated at 37° C. for 2 hours. Then 1*10⁶ PBMCs were stimulated with 1*10⁵ S20 loaded T2 cells for 14 days, and the medium was supplemented with a final concentration of 10 ng/mL IL-7 (Peprotech, Hamburg, Germany) and IL-15 (Peprotech, Hamburg, Germany) at a final concentration of 10 ng/mL, and aldesleukin (Novartis Pharmaceuticals) at a final concentration of 50 U/mL.

T cells were stained with HLA-A*02-S20 multimers, and CD8⁺ T cells bound to HLA-A*02-S20 multimers were isolated and enriched by flow cytometry. T cells were further screened for S20 epitope-specific clones. Extract the S20 epitope-specific clones for RNA sequencing to acquire the sequences of both chains of TCR, and construct HBV S20-specific TCR library. Three clones, A01/B01/C01, were selected, whose TCRs have high affinity and do not require modification of the variable regions (e.g., affinity maturation). The amino acid sequences of CDR1, CDR2, CDR3, TCRα variable regions and TCRβ variable regions of the α and β chains corresponding to A01/B01/C01 are showed in Table 1.

TABLE 1
Amino acid sequences of CDRs, TCRα variable regions
and TCRβ variable regions of A01/B01/C01.

| TCR fragments | A01 | B01 | C01 |
|---|---|---|---|
| αCDR1 | SEQ ID NO: 1 | SEQ ID NO: 13 | SEQ ID NO: 1 |
| αCDR2 | SEQ ID NO: 2 | SEQ ID NO: 14 | SEQ ID NO: 2 |
| αCDR3 | SEQ ID NO: 3 | SEQ ID NO: 15 | SEQ ID NO: 24 |
| βCDR1 | SEQ ID NO: 4 | SEQ ID NO: 16 | SEQ ID NO: 4 |
| βCDR2 | SEQ ID NO: 5 | SEQ ID NO: 17 | SEQ ID NO: 5 |
| βCDR3 | SEQ ID NO: 6 | SEQ ID NO: 18 | SEQ ID NO: 25 |
| TCRα variable | SEQ ID NO: 7 | SEQ ID NO: 19 | SEQ ID NO: 26 |
| region | | | |
| TCRβ variable | SEQ ID NO: 8 | SEQ ID NO: 20 | SEQ ID NO: 27 |
| region | | | |

Further, at least one additional disulfide bond was introduced into the TCR constant region to improve stability and reduce mismatch between internal and external TCR chains. On the other hand, the expression of TCR gene could be successfully improved by codon optimization. The amino acid sequences of the modified TCRα chain and TCRβ ˜chain, and the coding sequences of the modified TCRα variable region, TCRβ variable region, TCRα chain and TCRβ chain of A01, B01 and C01 are shown in Table 2. The TCR α and β chains are linked by a P2A self-cleaving peptide element to ensure that each transduced cell expresses the same level of α and β chains. The amino acid sequences of the single-chain TCR molecules corresponding to A01, B01 and C01 are shown in SEQ ID NO: 12, SEQ ID NO: 23 and SEQ ID NO: 30; the coding sequences are shown in SEQ ID NO: 37, SE ID NO: 42 and SE ID NO: 47, respectively.

TABLE 2
The amino acid and coding sequence of
modified A01/B01/C01 TCR and fragment

| TCR | A01 | B01 | C01 |
|---|---|---|---|
| TCRα | SEQ ID NO: 10 | SEQ ID NO: 21 | SEQ ID NO: 28 |
| TCRβ | SEQ ID NO: 11 | SEQ ID NO: 22 | SEQ ID NO: 29 |
| TCR single | SEQ ID NO: 12 | SEQ ID NO: 23 | SEQ ID NO: 30 |
| chain | | | |
| TCR Vα | SEQ ID NO: 33 | SEQ ID NO: 38 | SEQ ID NO: 43 |
| coding | | | |
| sequence | | | |
| TCR Vβ | SEQ ID NO: 34 | SEQ ID NO: 39 | SEQ ID NO: 44 |
| coding | | | |
| sequence | | | |
| TCRα chain | SEQ ID NO: 35 | SEQ ID NO: 40 | SEQ ID NO: 45 |
| coding | | | |
| sequence | | | |
| TCRβ chain | SEQ ID NO: 36 | SEQ ID NO: 41 | SEQ ID NO: 46 |
| coding | | | |
| sequence | | | |
| Single chain | SEQ ID NO: 37 | SEQ ID NO: 42 | SEQ ID NO: 47 |
| TCR coding | | | |
| sequence | | | |

Clone the Target Gene into pCDH Plasmid:

In order to improve the expression efficiency of TCR gene, it is very important to select a suitable vector plasmid. Based on the original sequence of the public pCDH-EF1-MCS-T2A-copGFP plasmid (the information of the restriction sites on the plasmid is shown in Table 3), we replaced the ampicillin resistance gene with the kanamycin resistance gene and carried out the total plasmid gene synthesis. Then design primers for both ends of the target gene to introduce EcoRI and SalI restriction sites to both ends of the target gene. PCR amplification was performed of the target gene (A01/B01/C01 single-stranded TCR coding sequence) to obtain an amplified product with a length of about 1800 bp. The electrophoresis band of target gene was purified and ligated into linearized pCDH-EF1-MCS-T2A-copGFP plasmid by double digested with EcoRI and SalI. The ligated product was transformed with Stbl3 competent cells, and single clones were picked and cultured. The target plasmids were extracted, then identified by double enzyme digestion, electrophoresis and sequencing. The target construct includes 5′LTR, HIV-1 Ψ, RRE, cPPT/CTS, EF-1α core promoter, WPRE, 3′LTR-SIN and other main functional elements (the key functional elements and positions of pCDH plasmid are shown in Table 4).

TABLE 3
Summary table of restriction site information of shuttle plasmids

| Position of enzyme digestion | restricted enzyme | recognition site |
|---|---|---|
| 3,090 | NheI | GCTAGC |
| 3,096 | EcoRI | GAATTC |
| 4,920 | SalI | GTCGAC |
| 5,521 | KpnI | GGTACC |

TABLE 4
Key functional elements and positions of pCDH plasmids

| NO. | elements | positions |
|---|---|---|
| 1 | 5′LTR | 837 . . . 1,017 |
| 2 | HIV-1 Ψ | 1,064 . . . 1,189 |
| 3 | RRE | 1,682 . . . 1,915 |
| 4 | cPPT/CTS | 2,411 . . . 2,527 |
| 5 | EF-1α core promoter | 2,570 . . . 2,781 |
| 6 | WPRE | 4,926 . . . 5,514 |
| 7 | 3′LTR | 5,588 . . . 5,821 |

### Example 2: Lentivirus Package

The lentiviral vector used in the A01/B01/C01 TCR-T cells was the third-generation “self-inactivating (SIN)” lentiviral vector derived from HIV-1 with a VSV-G pseudoenvelope, which was loaded with nucleic acids encoding targeting HBsAg-specific T-cell receptors (HBsAg TCRs). The lentiviral vector had only infective activity and no replication ability, the particle diameter was about 80-120 nm, and the shape was roughly spherical or icosahedral symmetrical structure. The outer membrane of the virus was a lipid-like envelope embedded with the VSV-G envelope protein. Inward was a spherical matrix (Matrix) formed by the protein p17, and a semi-conical capsid (Capsid) formed by the protein p24. There were RNA nucleic acid information comprising TCR encoding sequences in the capsid.

The functions of each element of the third-generation self-inactivating lentiviral vector are described as follows:

At both ends of the TCR gene are truncated/chimeric long terminal repeats, Δ5′LTR and Δ3′LTR, respectively. Among them, the U3 is removed from Δ5′LTR and replaced by the enhancer and promoter of respiratory syncytial virus pneumonia (RSV). The replication of the vector is no longer dependent on the “tat”. There is no priming/enhancing activity anymore after the U3 being removed from Δ3′LTR. And make it to be a self-inactivating vector.

The function of RRE: A cis-acting element of Rev that promotes the transport of large un-spliced mRNA molecules from the nucleus to the cytoplasm.

The function of cPPT: improving the transduction efficiency of the vector.

The function of EF-1a promoter: regulating the initiation time and degree of gene expression (transcription).

The function of WPRE: It can up-regulate the polyadenylation of transcripts, promote the nuclear export of transcripts, and improve the expression efficiency of target genes.

The lentiviral vector is obtained by transient transfection 293T cells with four-plasmid system of the third-generation, which consists of three packaging plasmids (also known as “helper plasmids”) and a shuttle plasmid.

The packaging plasmid pGagPol-KanR encodes the viral structural protein Gag and the reverse transcriptase Pol, the former forms the core structure of the virus, and the latter is necessary for RNA reverse transcription and integration.

The plasmid pRev-KanR encodes Rev protein, which binds to RNA to promote mRNA transport and protein expression.

The plasmid pVSV-G-KanR encodes the vesicular stomatitis virus envelope protein VSV-G, which replaces the HIV virus envelope protein, enabling the lentiviral vector to infect cells from almost all tissues and improving the stability of the lentiviral particles.

The D10 cell complete medium preparation: DMEM, 10% FBS (v/v), 1% Sodium Pyruvate, placed in a 4° C. refrigerator for later use.

Day 0: The 293T cells used were less than 20 generations, and the cells did not overfill the dish; 2*10⁷ cells were placed in a 150 mm dish, 20 mL of D10 medium was added with sufficient mixing and cultured at 37° C. overnight.

Day 1: 293T cells were transfected when the confluence reached 60-80%, and the time from the cells being planked to transfection was not more than 24 hours.

Plasmid complexes of lentiviral packaging were prepared according to Table 5.

TABLE 5
Materials required for plasmid complexes

| shuttle plasmid | RRE | REV | VSVG | PEIpro | OptiMEM |
|---|---|---|---|---|---|
| 18 μg | 10 μg | 7 μg | 7 μg | μL | 1 mL + 1 mL |

While gently vortexing the plasmid, PEIpro was added dropwise with sufficient mixing, then, rest at room temperature for 15 min to form plasmid-PEI complex. The complex was slowly added to a 150 mm dish of 293T cells with sufficient mixing before incubation at 37° C. for 6 hours in a carbon dioxide incubator.
Day 1: The transfection medium of the 293T cells (6 hours later) were gently replaced by complete D10 medium.
Day 3: The viral supernatant of transfection for 48 hours was collected, and temporarily stored in a 4° C. refrigerator. Then, 20 mL of D10 medium was added.
Day 4: The viral supernatant of transfection for 72 hours was collected and mixed with the viral supernatant of transfection for 48 hours before centrifugation at 3000 g for 10 min at 4° C. The debris was removed through a 0.45 μm filter and the supernatant was retained. The 100K ultrafiltration cups were used for virus concentration.

After centrifugation at 3000 g to the desired volume of virus concentration at 4° C., the centrifuge device was taken out, and the filter cup was separated from the filtrate collection cup. The filter cup was put upside down on the sample collection cup. The virus concentrate in the sample collection cup was collected after centrifugation at 1000 g for 2 min at 4° C. and stored below −70° C. after packing.

Detection of virus solution titer: 1*10⁵ cells/hole Jurkat cells were seeded into the 24-well plate. A certain amount of virus concentrates diluted in a gradient manner were added to the Jurkat cells. After culturing for 72 hours, the virus titer was detected by flow cytometry.

### Virus Titer Detection:

Flow cytometry buffer preparation: DPBS, 2% FBS, stored at 4° C. for later use.

The supernatant was abandoned of 1*10⁶ Jurkat cells in each group after centrifugation at 400 g for 5 min and washed twice with flow buffer.

PE Dextramer HBV-S20 cells were diluted with flow buffer at a ratio of 1:100. 100 μL antibody diluent was added to each sample, the flow buffer was added to wash the cells after incubation at 4° C. for 30 min in the dark, then the supernatant was discarded after centrifugation at 400 g for 5 min; and repeated the process twice.

The cells were suspended with 100 μL flow cytometry buffer and detected by flow cytometry.

Vector infection titer (TU/mL)=Number of cells per well×positive rate (%)×dilution ratio/titrated volume (mL)

The results were shown in FIG. 3. The lentivirus titers of A01, B01 and C01 TCR were all above 1*10⁸ TU/mL, indicating that all three HBV S20 TCRs can successfully package the virus with high titers.

### Example 3: Preparation of HBV S20 TCR-T

Preparation of T cell culture medium: PRIME-XV-T cell CDM, 400 IU/mL IL-2.

Preparation of T cell cryopreservation: 75% CS10+25% HSA.
Day 0: Isolated pure CD3⁺ T cells from apheresis, adjusted the cell concentration to 1*10⁶ cells/mL with T cell medium. The activator was added according to transact (CD3/CD28 microspheres): cell suspension=1:30 with sufficient mixing, and then interleukin 2 with the final concentration of 400 IU/mL was added to stimulate the cells 24 hours before virus infection.
Day 1: The density of T cells was adjusted to 5*10⁵ cells/mL, and virus solution was added;
Day 2-11: After the infection, observed the cell state every day, and T cell culture medium containing 400 IU/mL of IL-2 was added in a timely manner to maintain the density of T cells at 5*10⁵/mL to expand the cells;
Day 12: Harvest cells by centrifugation at 300 g for 5 min, the cells were washed by physiological saline solution containing 5% human serum albumin, frozen with a special freezing solution for T cells according to the appropriate density and were stored in the liquid nitrogen after being frozen by a programmed cooler.

### Example 4: The MOI Detection of S20 TCR-T and TCR-Specific Expression

### HBV S20 TCR MOI Detection

Flow buffer (FACS buffer) preparation: DPBS, 2% FBS (v/v), stored at 4° C. for later use.

TCR-T cells infected by A01, B01, C01 lenti-virus and NT cells (control group) were harvested and washed by FACS buffer, then stained with PE Dextramer HBV-S20 and incubated at 4° C. in dark for 30 min. The cells were washed with FACS buffer and analyzed by flow cytometry.

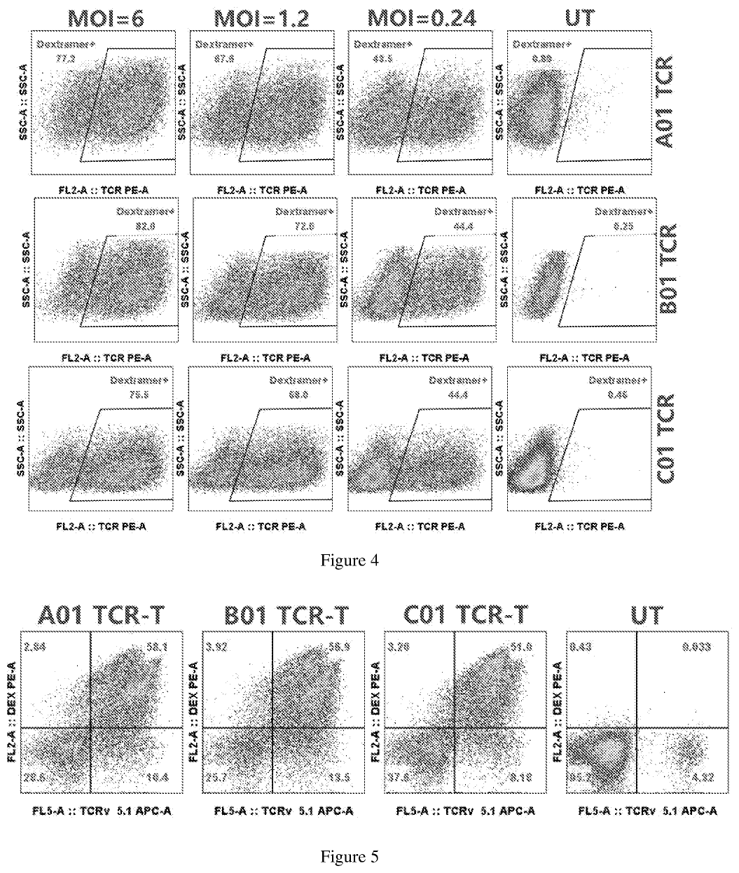

As shown in FIG. 4, the expression level of A01, B01, and C01 TCR-T were consistent with each other. When the MOI_{HBV S20 TCR}=1.2 in each group, the TCR-T positive rate was above 60%, indicating that all the three S20 TCR clones were stably expressed in TCR-T cells infected by gradient MOI.

### Specificity Expression Detection of HBV S20 TCR-T

The prepared cells and NT cells (control group) from each group of A01 TCR-T, B01 TCR-T and C01 TCR-T, were harvested and washed with FACS buffer, then stained with PE Dextramer HBV-S20 and APC TCR vβ 5.1 and incubated at 4° C. in dark for 30 min. The cells were washed with FACS buffer and applied to flow cytometry.

As shown in FIG. 5, a the mispairing rates of A01, B01, and C01 TCR-T were low, and the specific expression reached more than 50%, indicating that the three clones of HBV S20 TCR-T can be specifically expressed with low mispairing rate.

### Example 5: Cytolysis and Cytoline Analysis of S20 TCR-T on HepG2-LMS-LG Cell

The M10 complete medium preparation: DMEM, 10% FBS(v/v), 1% Sodium Pyruvate, 1% HEPES, 1% NEAA, stored at 4° C. for later use.

### Preparation of HepG2-LMS-LG Target Cells

3 wells of HLA-A02 subtype HepG2 cells were seeded in collagen precoated 24-well plate at the amount of 1*10⁵ cells/well; then 25 μL, 5 μL, 1 μL LMS-LG lentiviral vectors which encoded full-length HBsAg protein (including FLLTRILTI sequence) fused with luciferase and GFP protein were added to prepare the target HepG2-LMS-LG cell. When the cell confluence reached 90%, they were transferred to 6-well plate to expand. Flow detection was performed when the cell confluence reached about 90% again. The cells with the GFP positive rate greater than 95% were selected for expansion culture, and the seed cell bank of the target cell was established.

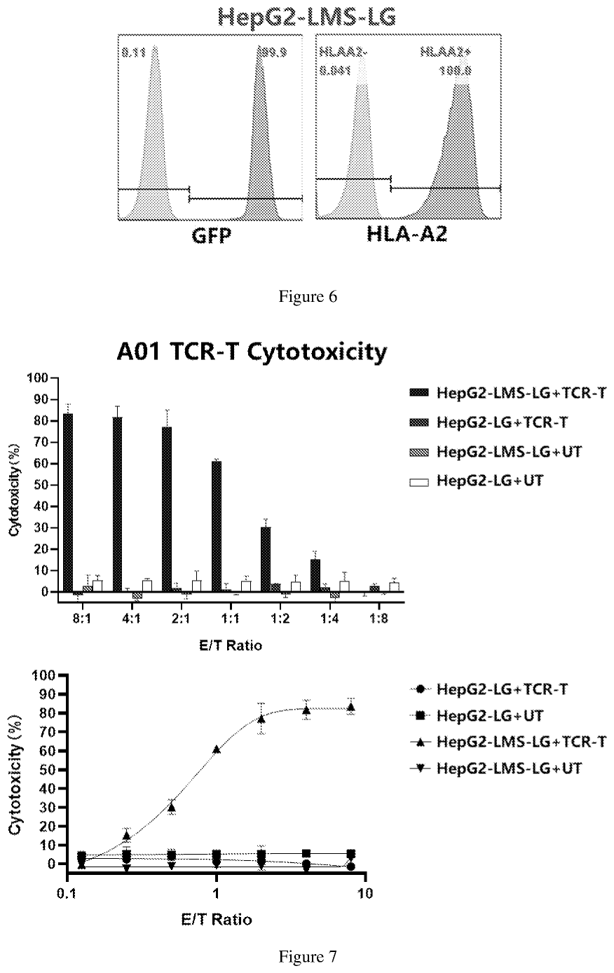

As shown in FIG. 6, the positive rate of HepG2-LMS-LG in the target cells was greater than 99% and the expression was uniform, indicating that the target cells were successfully constructed.

### Killing of A01 TCR-T on HepG2-LMS-LG Target Cells (Chemiluminescence Method)

D0: The hepG2-LMS-LG cells were seeded into collagen pre-coated 96-well plate at the density of 1*10⁴/well, 100 μL/well. 100 μL A01 TCR-T cells were added at the (effector to target) E:T ratio at 8:1, 4:1, 2:1, 1:1, 1:2, 1:4, 1:8, respectively; 100 μL culture medium was added to the wells with A01 TCR-T cells; Then cultured in 37° C. for 24 hours.
D1: 50 μL ONE-Glo™ Luciferase Assay System was added to each well with sufficient mixing and applied to microplate reader.

Calculation method: Killing efficiency=(1−average value of effector and target cells/average value of target cells)×100%.

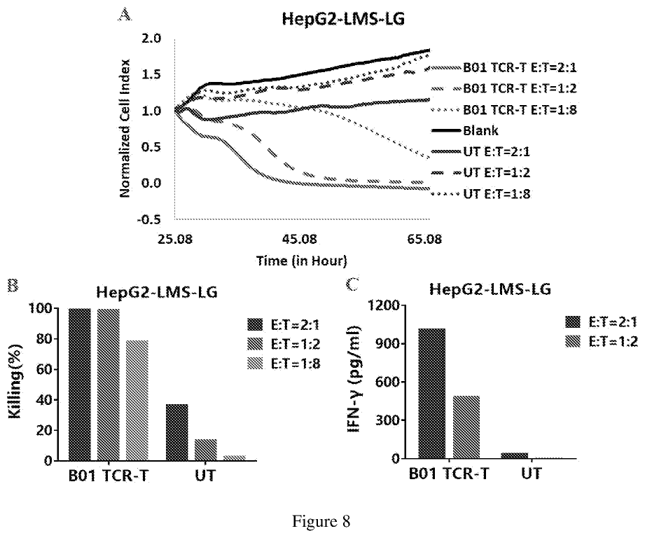

As shown in FIG. 7, the killing efficiency of A01 TCR-T on target cells reached more than 80% at the E:T ratio of 8:1 and 4:1. As the E:T ratio decreased, the killing efficiency decreased; the killing effect could also be detected until the E:T ratio was 1:4. As the negative control, the killing function was not observed in HepG2 vs A01 TCR-T and UT vs HepG2-LMS-LG cells, indicating that A01 TCR-T could specifically kill HBsAg-positive target cells.

Killing function of B01/C01 TCR-T on HepG2-LMS-LG cells (Real Time Cellular Analysis, RTCA)
D0: The cell density of the HepG2-LMS-LG cells was adjusted to 4*10⁵/mL after digestion. 50 μL/well M10 medium was added into the holes of 96-well plate pre-coated with collagen for priming the instrument baseline determination of the RTCA. Then 50 μL of cells (4*10⁵/mL) were added into each well. After resting for about 5 min, the growth curve was continuously detected for about 16 hours.
D1: The TCR positive rate and cell viability rate of B01/C01 TCR-T cells were detected.

Prepared effector cells at the density of 8*10⁵ positive cells/mL, 2.0*10⁵/mL, 0.5*10⁵/mL, then 50 μL/well of effector cells were added at the effector-target ratio 2:1, 1:2, 1:8, and the killing curve was continuously monitored.
D3: 72 hours later, RTCA monitoring was terminated for data copying and killing curve fitting, and the co-culture supernatant was collected for cytokine analysis.

The detection of cytokine was carried out according to the kit instructions:

The CBA kit (Human Th1/Th2 Cytokine Cytometric Bead Array Kit II, BD, 551809) was taken out, and balanced to room temperature.

2 mL of Assay Diluent was used to dissolve the standard substances to a concentration of 5000 pg/mL and equilibrate at room temperature for 30 min.

Standard preparation: The standard described above was marked as S1, the S2-S9 were diluted by 2 times in turn, and S10 was blank.

Configuring Human Th1/Th2 Cytokine Capture Beads Mixture, the microsphere solutions A1-A6 were vortexed thoroughly before mixing them at 1:1:1:1:1:1 ratio.

50 μL of Human Th1/Th2 Cytokine Capture Beads mixture was added into the 96-well U-shaped bottom plate, with the addition of 50 μL of Human Th1/Th2 PE Detection Reagent.

50 μL supernatant was added to each detection well after centrifugation at 400 g for 5 min, or 50 μL S10-S1 was added to the standard well.

Incubationat room temperature for 180 min in dark, 100 L wash buffer was added, and the supernatant was abandoned after centrifugation at 300 g for 5 min.

Resuspended with 100 L FACS buffer, and loaded the sample into flow cytometry for detection, and analyzed the results.

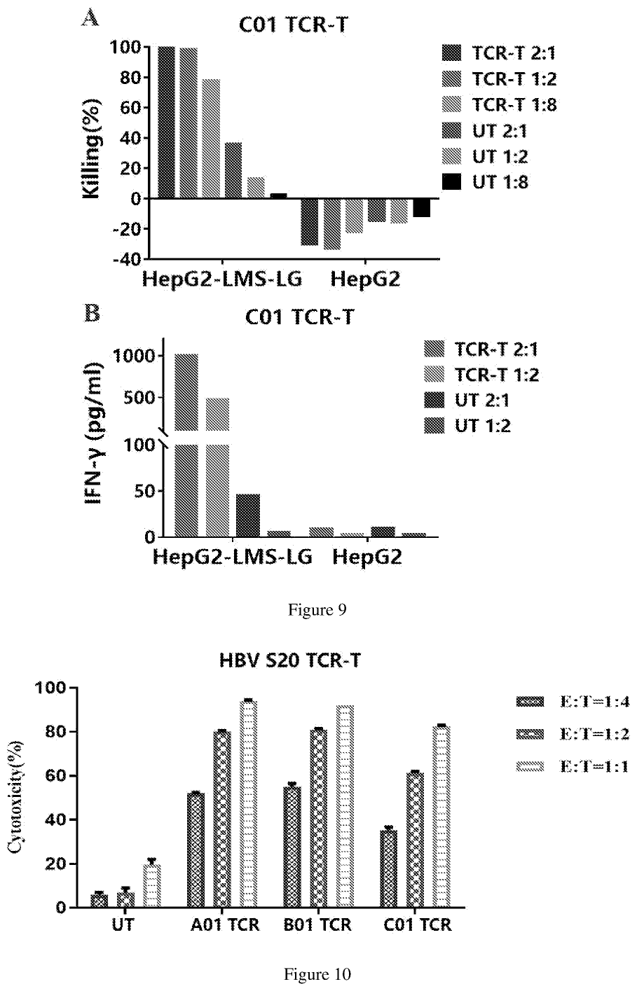

As shown in FIG. 9A, FIG. 9B, the killing ratios all reached more than 60% after 48 hours coculture with B01 TCR-T at the E:T ratio of 2:1, 1:2, 1:8, and the UT had no obvious function on target cells. The IFN-γ secretion of B01 TCR-T treated group was significant while the UT group was slight (see in FIG. 9C). The killing ratios also reached more than 60% after 48 hours coculture with C01 TCR-T at the E:T ratios of 2:1, 1:2, 1:8, and the UT observed no cytolysis function on target cells, and also C01 TCR-T had almost no function on negative cells. In terms of FIG. 10A the IFN-γ secretion of C01 TCR-T was obvious with target cells, but lower with the negative cells. In addition, UT treated group had no obvious cytokine secretion (see in FIG. 10B).

Killing function of A01/B01/C01 TCR-T on HepG2-LMS-LG cell
D0: The HepG2-LMS-LG cells were seeded into collagen pre-coated 96-well plate at the density of 1*10⁴/well, 100 μL/well; then 100 μL A01, B01, and C01 TCR-T cells were added at the ratio of E:T of 1:1, 1:2, 1:4; supplemented with 100 μL of T cell culture medium in the control wells, and co-cultured in 37° C. incubator for 24 hours.
D1: 50 μL ONE-Glo™ Luciferase Assay System was added to each well with sufficient mixing and applied to microplate reader for detection.

Calculation method: Killing efficiency=(1−average value of effector and target cells/average value of target cells)×100%.

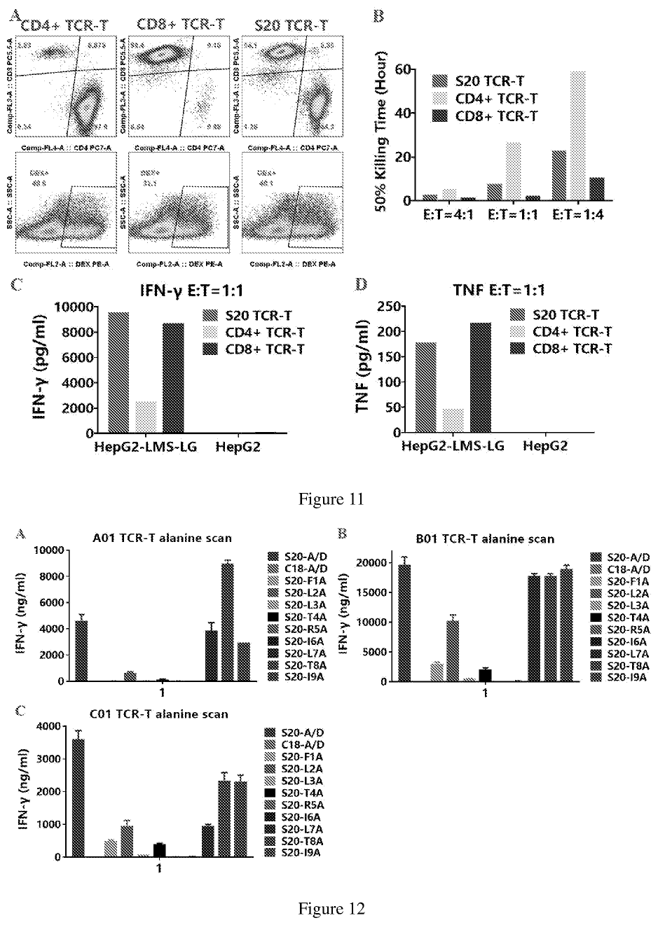

As shown in FIG. 11, A01, B01, and C01 TCR-T all showed significant specific killing effect on HepG2-LMS-LG in the dose-dependent manner, but had almost no killing effect on HBsAg-negative target cells.

### Example 6: Functionality of Different T Cell Subsets of HBV S20 TCR-T on Target Cells

CD4⁺ T cells are helper T lymphocytes whose main function is to enhance the anti-infection mediated by phagocytes and enhance the humoral immune response mediated by B cells. CD8⁺ T cells are suppressor/killer T lymphocytes whose main function is to specifically kill target cells directly. We investigated the anti-tumor activity of HBV S20 TCR-T by isolating CD4⁺/CD8⁺ cell subsets of HBV S20 TCR-T.

### Cell Sorting

25 μL of Dynabeads® CD4 and Dynabeads® CD8 positive magnetic beads were added to 1*10⁶ HBV S20 TCR-T cells, blown evenly with sufficient mixing, respectively, then transferred to a separation column and incubated at 2-8° C. for 20 min.

The separation column was put into the magnetic stand for 2 min. The supernatant in the tube was abandoned after the cells attached to the magnetic beads were adsorbed on the tube wall. The separation column was removed and 1 mL of washing solution (Buffer 1) was added to rinse 2-3 times, then placed on the magnetic stand again for 2 min. The above steps were repeated 5 times.

Resuspend Cells with 100 μL of Buffer2.

10 μL of DETACHaBEAD® was added before incubation at room temperature for 45 min with mixing gently.

The separation column was put into the magnetic stand for 1 min, and the T cells in the supernatant were transferred to a new test tube.

The cells were thoroughly washed with 4 mL Buffer2, and the supernatant was discarded by centrifugation at 400 g for 5 min.

The obtained high-purity CD4⁺ and CD8⁺ live cells without magnetic beads were used for subpopulation phenotyping by flow cytometry and subsequent functional experiments.

### Detection of Cell Subpopulation:

FACS buffer preparation: DPBS, 2% FBS, placed in 4° C. refrigerator for later use.

The sorted CD4⁺ and CD8⁺ cells were blown evenly and centrifugated at 400 g for 5 min with two times wash using FACS buffer.

PE-Cy7-CD4 (BIOLEGEND, 300512)/PerCP-Cy5.5-CD8 (BIOLEGEND, 301032)/PE Dextramer antibodies were diluted 1:100 with FACS buffer, and 100 μL of detecting antibody was added to each sample, then incubated at 4° C. in dark for 30 min. Samples were washed twice with FACS and centrifugated at 400 g for 5 min buffer before analysis.

Cells were resuspended in 100 μL FACS buffer and detected by flow cytometry.

As shown in FIG. 11A, the purity of the sorted CD4⁺ and CD8⁺ cells were over 95%, and the positive rate of CD4⁺ cells was slightly higher than that of CD8⁺ cells.

### Killing Function of S20 TCR-T on HepG2-LMS-LG Cells (RTCA)

Target cell complete medium (M10): DMEM, 10% FBS, 1% Sodium Pyruvate, 1% HEPES, 1% NEAA, placed in a 4° C. refrigerator for later use.

T cell complete medium (TCM): IL-2 was added to an appropriate amount of PRIME-XV culture in a 50 mL centrifuge tube at a final concentration of 400 IU/mL with sufficient mixing and stored at 2-8° C. for later use.

D0: The cell density of the HepG2-LMS-LG cells with good growth status was adjusted to 4*10⁵/mL after digestion. 50 μL/well M10 medium was added into the holes of a 96-well coated with collagen plate for the baseline determination of RTCA. Then 50 μL of cells (4*10⁵/mL) were added into each well. After resting for about 5 min, the growth curve was continuously detected for about 16 hours.
D1: The TCR positive rate and cell viability rate of CD4⁺ and CD8⁺ sorted TCR-T cells were detected, and the unsorted TCR-T cells were served as controls. 50 μL/well of effector cells were added at the effector-target ratios of 4:1, 1:1, 1:4, and the killing curve was continuously detected through the machine.
D3: 72 hours later, RTCA monitoring was terminated for data copying and killing curve fitting, and the co-culture supernatant was collected for cytokine analysis.

Cytokine detection was carried out according to the detection procedure of cytokines in Example 5.

As shown in FIG. 11B, CD8⁺ cells exhibited excellent killing ability on tumor cells at the three effector-target ratios, and the half-killing time of CD8⁺ cells was only half of the control group. Under the condition of high target ratio, CD4⁺ cells also had the good killing effect on target cells. In the case of effector-target ratio of 1:1, the cytokine release level of CD8⁺ cells were the same as that of the control group, but higher than that of the CD4+ cells group (see in FIG. 11C, FIG. 11D).

In conclusion, these results suggested that CD8⁺ TCR-T cells have stronger killing and cytokine release ability on tumor cells than CD4⁺ TCR-T cells.

### Example 7: Detection of Cross-Reactivity of A01/B01/C01 TCR-T to Human Polypeptide Database

Due to the sequence diversity of TCRs, epitopes and MHC/HLA molecules, TCRs may have cross-reactivity leading to potential off-target toxicity. The Alanine Scanning Peptide Library can be used to identify specific amino acid sites that are closely related to polypeptide function, stability and conformation. Each amino acid residue in the HBV S20 epitope peptide individually mutated to alanine for testing the cross-recognition ability of HBV S20 TCR-T on the mutated epitope.

Identification of key amino acids recognized by HBV S20 TCR-T

The target cell complete medium (R10): prepared at the ratio of RPMI 1640:FBS=10:1 with sufficient mixing and stored at 2-8° C. for later use.

The T cell complete medium (TCM): IL-2 was added to an appropriate amount of PRIME-XV culture in a 50 mL centrifuge tube at a final concentration of 400 IU/mL with sufficient mixing and stored at 2-8° C. for later use.

Peptide preparation: the positive control S20-A/D (No. 1), the amino acid sequence of No. 1 is FLLTRILTI. The negative control C18-A/D (No. 2), the amino acid sequence of No. 2 is FLLTKILTI. The S20 mutant polypeptides (No. 3-11), which mutated F1A, L2A, L3A, T4A, R5A, I6A, L7A, T8A and I9A compared to the amino acid sequence of FLLTRILTI, respectively. 2 mg of each peptide was dissolved in 170 L DMSO, the final concentration was 10 mM. 3 μL of 10 mM solution was diluted with 297 μL TCM to 100 μM before 10-fold dilution to 10 μM.

Target cell preparation: The T2 cells were harvested and resuspended in R10 medium. The cell viability and density were measured. 1.8*10⁶ T2 cells were resuspended with 6 mL TCM medium according to the counting results. 100 μL/well of T2 cells (3*10⁴/well) were added in the 96-well U-bottom plate. The corresponding peptide solutions were added into the plate at a final concentration of 1 μM. The effector cells were added after the 96-well U-bottom plate was put into 37° C. incubator for 2 hours.

Preparation of the effector cells: The viability and density of HBV S20 TCR-T cells were measured. 2.6*10⁶ HBV S20 TCR-T cells were resuspended with 3 mL TCM medium, then the density of positive cells was adjusted to 6*10⁵/mL. 50 μL/well of effector cells were added to each well and the plate was put in 37° C. incubator. The cytokine level in the supernatant was measured by flow cytometry after co-incubating for 24 hours.

Cytokine detection was carried out according to the procedure in Example 5. The results showed that the specially recognizing function of HBV S20 TCR-T on peptides with amino acid mutation at positions 3/4/5/6 significantly diminished, and the secretion of cytokines was undetectable or much lower than the group of the original S20 peptide (FIG. 12, the histogram from the left to the right represented the results of peptides No. 1-11 in turn). The key amino acid motifs (3/4/5/6, LTRI) of the HBV S20 TCR-T recognition epitope were identified. The 4 amino acid residues were considered to play a key role in direct binding or controlling the spatial structure in the binding to S20 TCR. These results provided a guidance for computer predictive analysis to evaluate the cross-reactivity of other similar human self-antigen peptides, and further evaluating the specificity and safety of the epitope recognition of HBV S20 TCR-T.

We obtained 14 peptide sequences with 6 amino acids identical to the S20 epitope peptide, based on computer prediction and sequence alignment in database, and BLAST sequence alignment of the human peptide library. No human peptides with more than 6 amino acids identical to S20 were found. T2 was used to load different concentrations of peptides and then incubated with HBV S20 TCR-T to detect cytokine secretion.

Peptide preparation: The positive control S20-A/D (No. 1), the negative control C18-A/D (No. 2), and 14 peptide sequences with 6 amino acids identical to the S20 epitope peptide (No. 14-27). 2 mg of each peptide was dissolved in 200 L DMSO, the final concentration was 10 mg/mL. 10 μL of 10 mg/mL solution was diluted with 90 μL TCM to 1 mg/mL before 10-fold gradient dilution to 100 ng/mL.

Target cell preparation: The supernatant was removed after centrifugation of T2 cells in good growth status after several times of passages at 500 g/5 min. The cells were resuspended in RIO medium. The cell viability and density were measured. 1.8*10⁶ T2 cells were resuspended with 9 mL TCM medium according to the counting results. 100 μL/well of T2 cells (2*10⁴ well) were added in the 96-well U-bottom plate. The corresponding peptide solutions were added into the plate at a final concentration of 0.1 μg/mL and 1 μg/mL. The effector cells were added after the 96-well U-bottom plate was put into an incubator for incubation 2 hours at 37° C.

Preparation of the effector cells: The T2 cells were harvested and resuspended in RIO medium. The cell viability and density were measured after the HBV S20 TCR-T cells blown evenly. 4.4*10⁶ HBV S20 TCR-T cells were resuspended with 4.5 mL TCM medium according to the counting result, then the density of positive cells was adjusted to 1*10⁶/mL. 50 μL/well of effector cells were seeded into each well and the plate was put into 37° C. incubator. The cytokine level in the supernatant was measured by flow cytometry after co-incubating for 24 hours.

Cytokine detection was carried out according to the procedure in Example 5.

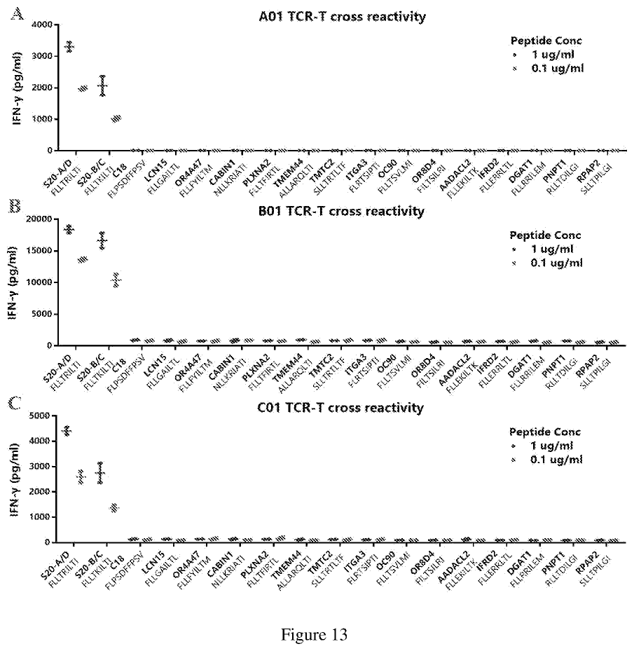

As shown in FIG. 13, A1/B01/C01 TCR-T had no cross-reactivity to the fourteen polypeptides.

In conclusion, we demonstrated that the key amino acid motif recognized by HBV S20 TCR-T is at the positions of 3 to 6 (LTRI). If the amino acid residues in this region were changed, the recognition function of TCR for mutant polypeptides significantly diminished. 14 human peptide sequences with 6 amino acids identical to the S20 epitope peptide were obtained by comparing the human peptide library through the bioinformatics prediction algorithm. No cross-reaction of HBV S20 TCR-T to these human autoantigenic peptides was observed by T2 cell loading method, indicating that the potential off-target toxicity risk of HBV S20 TCR-T against human endogenous antigens is very low.

### Example 8: Detection of the Binding Ability of A01/B01/C01 TCR-T to S20 with Different Genotypes

T cells recognize tumor antigens, mainly through the TCR recognition of tumor antigens and HLA-peptide complexes on the surface of target cells. The activation signals were transduced through the specific binding of TCR-T with tumor antigens, thereby producing targeted killing functions on tumor cells. To study the function of HBV S20 TCR-T on HBV with different genotypes, T2 cells were loaded with different concentrations of S20-gt A/D (genotype A or D with amino acid sequence FLLTRILTI) or S20-gt B/C (genotype B or C with amino acid sequence FLLTKILTI).

The target cell complete medium (R10): prepared at the ratio of RPMI 1640:FBS=10:1 with sufficient mixing and stored at 2-8° C. for later use.

The T cell complete medium (TCM): IL-2 was added to an appropriate amount of PRIME-XV culture in a 50 mL centrifuge tube at a final concentration of 400 IU/mL with sufficient mixing and stored at 2-8° C. for later use.

Peptide preparation: 2 mg of S20-AD, S20-BC, C18-AD peptide was dissolved in 170 μL DMSO, respectively; the final concentration was 10 mM. 3 L of 10 mM solution was diluted with 297 μL TCM to 100 M before 10-fold gradient dilution to 10 μM.

Target cell preparation: The T2 cells were harvested and resuspended in R10 medium. The cell viability and density were measured. 2.5*10⁶ T2 cells were resuspended with 5 mL TCM medium according to the counting results. 100 μL/well of T2 cells (5*10⁴/well) were added in the 96-well U-bottom plate; 100 μL TCM was added to each well of the negative control group. The corresponding peptide solutions were added into the plate at a final concentration of 10⁻⁵ M-10⁻⁹ M. The effector cells were added after the 96-well U-bottom plate was put into 37° C. incubator for 2 hours.

Preparation of the effector cells: The T2 cells were harvested and resuspended in RIO medium. 3.6*10⁶ HBV S20 TCR-T cells were resuspended with 2.5 mL TCM medium according to the counting result, then the density of positive cells was adjusted to 1*10⁶/mL. 50 μL/well of effector cells were seeded into each well and the plate was put into 37° C. incubator. The cytokine level in the supernatant was measured by flow cytometry after co-incubating for 24 hours.

Cytokine detection was carried out according to the procedure in Example 5.

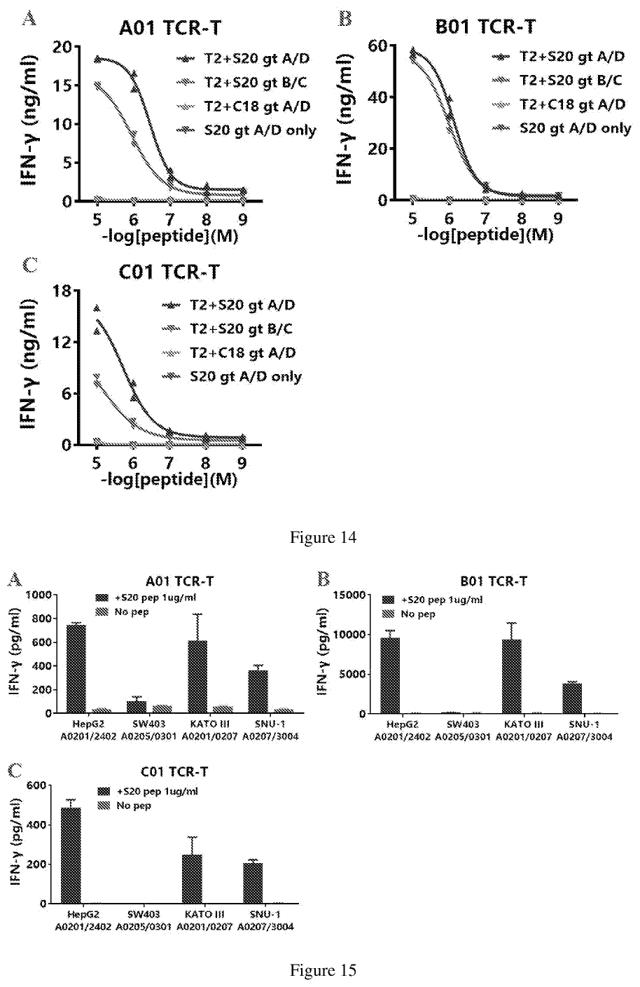

As shown in FIG. 14, A01/B01/C01 TCR-T had significant cytokine secretion on T2 cells loaded with HBV S20 gt A/D polypeptide or S20 gt B/C polypeptide. The secretion of IFN-γ reached more than 5 ng/mL when the concentration of loaded polypeptide reached 10⁻⁵ M. It had no function on C18-27 gt A/D. And the A01/B01/C01 TCR-T cytokine was secreted in a S20 dose-dependent manner.

In conclusions, HBV S20 TCR-T has obvious functions on HBV with different genotypes, HBV S20 TCR-T can cover most HBV virus subtypes, and has good functions on several common genotypes.

### Example 9: A01/B01/C01 TCR-T Recognized Different Subtypes of HLA-A02

It is restricted by HLA subtypes when TCR specifically recognizes antigenic peptides presented by MHC molecules. HepG2 is human hepatoma cell line with HLA-A*02:01/24:02; SW403 is human hepatoma cell line with HLA-A*02:05/03:01; KATO III is human gastric cancer cell line with HLA-A*02:01/02:07; SNU-1 is human gastric cancer cell line with HLA-A*02: 07/30. After the cells above loaded with S20 epitope peptides, the ability to stimulate HBV S20 TCR-T to kill target cells and secrete cytokines was tested to evaluate the binding capacity of S20 epitope peptide-MHC complexes presented by target cells of different subtypes of HLA-A02 and HBV S20 TCR-T in vitro.

The target cell complete medium (R10): prepared at the ratio of RPMI 1640:FBS=10:1 with sufficient mixing and stored at 2-8° C. for later use.

The T cell complete medium (TCM): IL-2 was added to an appropriate amount of PRIME-XV culture in a 50 mL centrifuge tube at a final concentration of 400 IU/mL with sufficient mixing and stored at 2-8° C. for later use.

Peptide preparation: 2 mg of S20 peptide was dissolved in 200 L DMSO to achieve a final concentration of 10 mg/mL. 10 μL of solution was diluted with 90 μL TCM to 1 mg/mL before 10-fold gradient dilution to 1 μg/mL.

Target cell preparation: HepG2, SW403, KATO III, and SNU-1 cells were harvested and resuspended in RIO medium. The cell viability and density were measured. 8*10⁵ cells were resuspended with 4 mL TCM medium according to the counting results. 2*10⁴/well of target cells (100 μL/well) were added in the 96-well U-bottom plate; 100 μL TCM was added to T cell only group without peptides. The corresponding peptide solutions were added into each well at a final concentration of 1 μg/mL. The effector cells were added after the 96-well U-bottom plate was put into 37° C. incubator for 2 hours.

Preparation of the effector cells: the cell viability and density were measured after the HBV S20 TCR-T cells blown evenly. The density of cells was adjusted by TCM according to the counting and positive rate result. 50 μL/well of effector cells (E:T=2:1) were seeded and the plate was put into 37° C. incubator. The cytokine level was measured by flow cytometry after co-incubating for 24 hours.

As shown in FIG. 15, HLA-A*02:01 (HepG2) was used as the reference, there was significant cytokine secretion could be detected after the A01 TCR-T, B01 TCR-T and C01 TCR-T co-incubated with KATO III and SNU-1 loaded with S20 polypeptide under the restriction of HLA-A*02:07, but no related cytokine secretion could be detected after the A01 TCR-T, B01 TCR-T and C01 TCR-T co-incubated with target cells without S20 polypeptide loading. There was no cytokine secretion could be detected of SW403 with or without S20 polypeptide loading under the restriction of HLA-A*02:07.

The HepG2, KATO III and SNU-1 cells loaded with S20 epitope peptides under the restriction of HLA-A*02:01, or HLA-A*02:07 can effectively activate HBV S20 TCR-T to secrete related cytokines, showing the significant specific binding activity. However, due to the limitation of HLA subtype, SW403 (HLA-A*02:05) cells loaded with S20 epitope peptide, cannot activate the killing function of HBV S20 TCR-T. The cross-recognition activity of HBV S20 TCR-T against S20 epitope peptides presented by multiple HLA-A*02:01 and HLA-A*02:07 alleles means that there is wide applicable range patient population covered by the HBV S20 TCR-T.

### Example 10: In Vivo Pharmacodynamics and Pharmacokinetics Experiments of A01 TCR-T in HepG2-LMS-LG Xenograft Model

In order to understand the anti-tumor activity of A01 TCR-T on tumor cells in vivo, we used HBsAg⁺ liver cancer cells (HepG2-LMS-LG) transplanted immunodeficient mice (Shanghai Model Organisms Center, Inc.) to construct the mouse CDX model, which was developed to evaluate the tumor elimination effect of A01 TCR-T cells on liver cancer.

1*10⁷ tumor cells were inoculated subcutaneously in the right axilla of NPG mice on Day −6. On Day 0, the tumor size reached about 100 mm³, and the mice were divided into 6 groups of 10 mice each group. Among them, 4 groups received different doses of A01 TCR-T cell injections, namely the high-dose group (2*10⁷ cells/mouse), the middle-dose group (1*10⁷ cells/mouse), the low-dose group (0.5*10⁷ cells/mouse), and the very low dose group (0.2*10⁷ cells/mouse), respectively. The remaining two groups were used as control groups which were received cell cryopreservation (vehicle) and un-transfected T cells (UT) (1.5*10⁷ cells/mouse), respectively. Tumor and pharmacokinetic data were collected, and tumor size was measured twice a week.

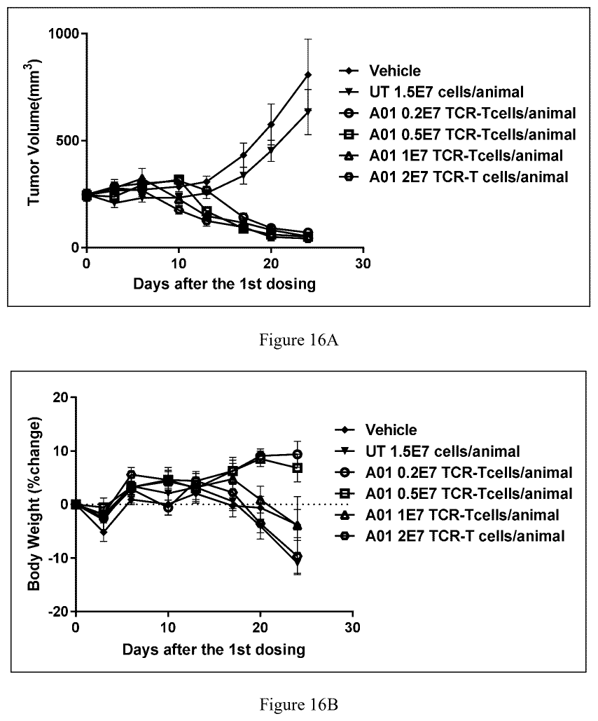

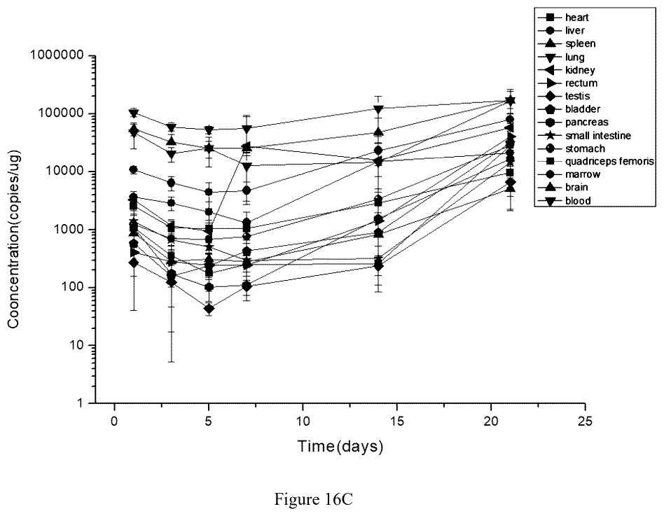

Compared with the control group, different doses of A01 TCR-T cells had a significant inhibitory effect on the growth of the tumor. The tumor volume in the four treated groups was significantly reduced (see in FIG. 16A), and the mice were well tolerated after intravenous injection and without loss of weight (see in FIG. 16B). The copy number of A01 TCR-T could be detected in all tissues 1 day after administration, then showed a trend of gradually decreasing, and it decreased to the lowest level 7 days after administration; but 14 days and 21 days after administration, A01 TCR-T cells re-expanded in tissues with rich blood flow such as liver, spleen, lung, and heart, and the number of A01TCRT cells peaked 21 days after administration (see in FIG. 16C).

Based on the above results, A01 TCR-T has anti-tumor activity on the HepG2-LMS-LG xenograft model, and the efficacy is positively correlated with the dose.

TCR-T cells in tissues, specifically such as the liver, spleen, lung, and heart with abundant blood flow, expanded significantly, and no other systemic toxicity related to the test product was found. Since the mononuclear cell infiltration may occur in multi-organ/tissue of animal model caused by graft-versus-host disease (GvHD), the test period was controlled at 21 days. Because HBV-S20 TCR-T cells were prepared in clinical trials using patient-derived autologous T cells, GvHD was avoided.

### Example 11: In Vivo Pharmacodynamics Experiments of B01 TCR-T in HepG2-LMS-LG Xenograft Model

We used immunodeficient mice (Shanghai Model Organisms Center, Inc.) transplanted with HBsAg⁺ liver cancer cells (HepG2-LMS-LG) to construct the mouse CDX model, which was developed to evaluate the tumor elimination effect of B01 TCR-T cells on liver cancer. 1*10⁷ tumor cells were inoculated subcutaneously in the right axilla of NPG mice on Day −6. On Day 0, the mice were divided into 6 groups of 10 mice each group. Among them, 4 groups received different doses of B01 TCR-T cell injections, namely the high-dose group (2*10⁷ cells/mouse), the middle-dose group (1*10⁷ cells/mouse), the low-dose group (0.5*10⁷ cells/mouse), and the very low dose group (0.2*10⁷ cells/mouse), respectively. The remaining two groups were used as control groups which were received cell cryopreservation (vehicle) and un-transfected T cells (Mock-T) (4.3*10⁷ cells/mouse), respectively. The tumor size was measured twice a week thereafter and the data were collected.

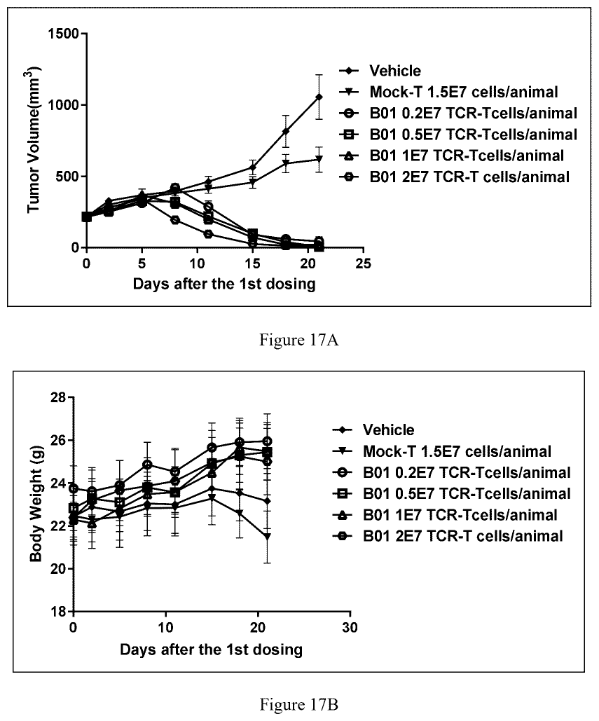

Compared with the control group, different doses of B01 TCR-T cells had a significant inhibitory effect on tumor growth, and the tumor volume in the four treated groups was significantly reduced (see in FIG. 17A). The tumor inhibition rates were all over 90%, and the mice were well tolerated after intravenous injection without weight loss (see in FIG. 17B).

In conclusion, there was obvious killing effect of B01 TCR-T on tumor cells in vivo with no observed adverse reactions.

### Example 12: In Vivo Pharmacodynamics Experiments of C01 TCR-T HepG2-LMS-LG Xenograft Model

We used immunodeficient mice (Shanghai Model Organisms Center, Inc.) transplanted with HBsAg⁺ liver cancer cells (HepG2-LMS-LG) to construct the mouse CDX model, which was developed to evaluate the tumor elimination effect of C01 TCR-T cells on liver cancer.

1*10⁷ tumor cells were inoculated subcutaneously in the right axilla of NPG mice on Day −6. On Day 0, the mice were divided into 4 groups of 5 mice in each group. Among them, 2 groups received different doses of C01 TCR-T cell injections, namely the high-dose group (2*10⁷ cells/mouse), and the low-dose group (5*10⁶ cells/mouse), respectively. The remaining two groups were used as control groups which were received cell cryopreservation (vehicle) and un-transfected T cells (Mock-T) (2.9*10⁷ cells/mouse), respectively. Tumor size was continuously monitored and data was collected thereafter.

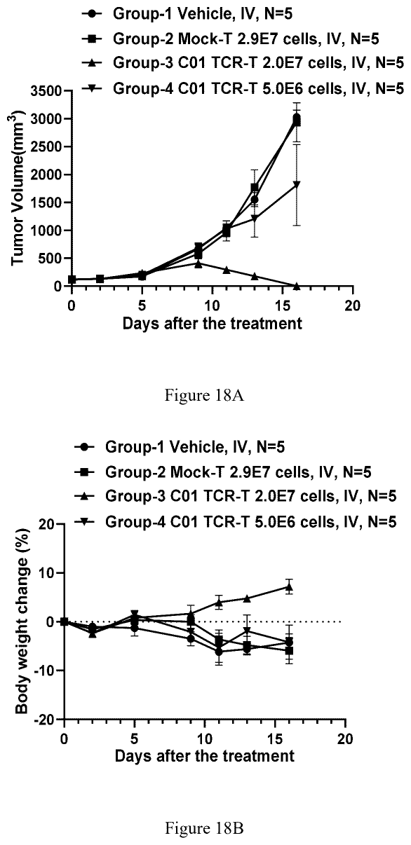

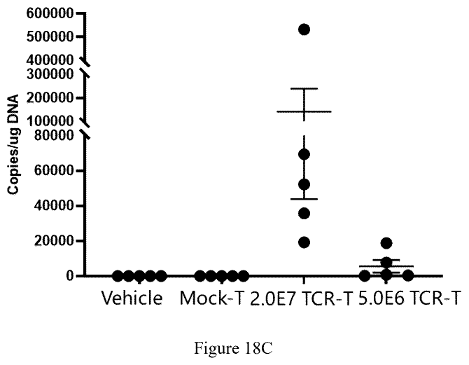

Compared with the control group, the high-dose C01 TCR-T cells had a more significant inhibitory effect on tumor growth than the low-dose group, and the tumor volume was significantly reduced (see in FIG. 18A). Mice in different dose groups were well tolerated after intravenous injection without loss of weight (see in FIG. 18B). Compared with the control group, a higher copy number of C01 TCR-T (the method of qPCR) could be detected in the animal tissues of the high-dose group, indicating the survival and expansion of TCR-T cells in the animals, and the degree of cell expansion showed a significant negative correlation with tumor size. It showed that the more obvious expansion of C01 TCR-T in mice, the stronger killing and inhibitory effect on tumor. (see in FIG. 18C).

In conclusion, there was a higher copy number of C01 TCR-T in vivo, and the C01 TCR-T had obvious killing effect on tumor cells in vivo with no obvious observed adverse reactions.

The present application has been introduced in detail above. The principles and implementations of the present application have been described with specific examples herein. The descriptions of the above embodiments are only used to help understand the methods and core ideas of the present application; for those skilled in the art, there may be changes in the specific embodiments and the scope of application according to the idea of the present application. Therefore, the contents of this specification should not be construed as the limitation of the application.

1. A T cell receptor (TCR) or its fragment thereof, comprising:
a TCRα chain variable region comprising a αCDR3 having the amino acid sequence:
αCDR3: ATDERDDMR (SEQ ID NO:3), or a variant thereof in which one or two amino acids are replaced with another amino acid;
and;
a TCRβ chain variable region comprising a βCDR3 having the amino acid sequence: βCDR3: ASSLNTEAF (SEQ ID NO:6) or a variant thereof in which one or two amino acids are replaced with another amino acid;
or;
a TCRα chain variable region comprising a αCDR3 having the amino acid sequence:
αCDR3: GADTSTDKLI (SEQ ID NO:15), or a variant thereof in which one or two amino acids are replaced with another amino acid;
and;
a TCRβ chain variable region comprising a βCDR3 having the amino acid sequence:
βCDR3: ASSHGGAYEQY (SEQ ID NO:18) or a variant thereof in which one or two amino acids are replaced with another amino acid;
or;
a TCRα chain variable region comprising a αCDR3 having the amino acid sequence:
αCDR3: ATDAYGQNFV (SEQ ID NO:24), or a variant thereof in which one or two amino acids are replaced with another amino acid;
and;
a TCRβ chain variable region comprising a βCDR3 having the amino acid sequence:
βCDR3: ASGSNTEAF (SEQ ID NO:25) or a variant thereof in which one or two amino acids are replaced with another amino acid.

2. The TCR or fragment according to claim 1, comprising:
a TCRα chain variable region comprising a αCDR1, a αCDR2 and a αCDR3 as shown in SEQ ID NO: 1, SEQ ID NO: 2 and SEQ ID NO: 3, respectively;
and;
a TCRβ chain variable region comprising a βCDR1, a βCDR2 and a βCDR3 as shown in SEQ ID NO: 4, SEQ ID NO: 5 and SEQ ID NO:6, respectively;
or a variant thereof in which one or two amino acids in one or more of the CDRs are replaced with another amino acid;
or;
a TCRα chain variable region comprising a αCDR1, a αCDR2 and a αCDR3 as shown in SEQ ID NO: 13, SEQ ID NO: 14 and SEQ ID NO: 15, respectively;
and;
a TCRβ chain variable region comprising a βCDR1, a βCDR2 and a βCDR3 as shown in SEQ ID NO: 16, SEQ ID NO: 17 and SEQ ID NO: 18, respectively;
or a variant thereof in which one or two amino acids in one or more of the CDRs are replaced with another amino acid;
or;
a TCRα chain variable region comprising a αCDR1, a αCDR2 and a αCDR3 as shown in SEQ ID NO: 1, SEQ ID NO: 2 and SEQ ID NO: 24, respectively;
and;
a TCRβ chain variable region comprising a βCDR1, a βCDR2 and a βCDR3 as shown in SEQ ID NO: 4, SEQ ID NO: 5 and SEQ ID NO: 25, respectively;
or a variant thereof in which one or two amino acids in one or more of the CDRs are replaced with another amino acid.

3. The TCR or fragment according to claim 1, wherein the TCRα chain variable region comprises the amino acid sequence that is at least 90% identical to any one of SEQ ID NO: 7, SEQ ID NO: 19 and SEQ ID NO: 26.

4. The TCR or fragment according to claim 1, wherein the TCRβ chain variable region comprises the amino acid sequence that is at least 90% identical to any one of SEQ ID NO: 8, SEQ ID NO: 20 and SEQ ID NO: 27.

5. The TCR or fragment according to claim 1, wherein the TCRα chain comprises amino acid sequence set forth in SEQ ID NO:10, SEQ ID NO:21 or SEQ ID NO: 28.

6. The TCR or fragment according to claim 1, wherein the TCRβ chain comprises amino acid sequence set forth in SEQ ID NO: 11, SEQ ID NO: 22 or SEQ ID NO: 29.

7. The TCR or fragment according to claim 1, which is capable of binding to a polypeptide of HBV surface antigen presented by HLA-A2.

8. The TCR or fragment according to claim 7, wherein the polypeptide comprises or consists of the amino acid sequence FLLTRILTI (SEQ ID NO: 31) or FLLTKILTI (SEQ ID NO: 32).

9. The TCR or fragment according to claim 1, wherein the C- or N-terminal of the α chain and/or β chain of TCR is combined with conjugates, and the described conjugates are a detectable marker, a therapeutic agent, a PK modifying moiety, or combination of them.

10. A nucleic acid molecule encoding the TCR molecule or fragment according to claim 1.

11. The nucleic acid molecule of claim 10, wherein the coding sequence of the variable region of the TCRα chain is set forth in SEQ ID NO: 33, SEQ ID NO: 38 or SEQ ID NO: 43;
and/or;
The coding sequence of the variable region of the TCR beta chain is set forth in SEQ ID NO: 34, SEQ ID NO: 39 or SEQ ID NO: 44.

12. The nucleic acid molecule of claim 10, wherein the coding sequence of the TCRα chain is set forth in SEQ ID NO: 35, SEQ ID NO: 40 or SEQ ID NO: 45;
and/or;
The coding sequence of the TCRβ chain is set forth in SEQ ID NO: 36, SEQ ID NO: 41 or SEQ ID NO: 46.

13. The nucleic acid molecule according to claim 10, wherein the nucleic acid molecule is comprised in an expression vector.

14. (canceled)

15. (canceled)

16. (canceled)

17. A method of treating diseases caused by HBV infection in a patient, comprising administering the TCR or fragment according to claim 1, or the nucleic acid molecule according to claim 10 to the patient.

18. (canceled)


SEQUENCE LISTING (47 sequences)

SEQ ID NO: 1 - protein, 5 aa, Synthetic construct
TSINN

SEQ ID NO: 2 - protein, 7 aa, Synthetic construct
IRSNERE

SEQ ID NO: 3 - protein, 9 aa, Synthetic construct
ATDERDDMR

SEQ ID NO: 4 - protein, 5 aa, Synthetic construct
SGHDY

SEQ ID NO: 5 - protein, 6 aa, Synthetic construct
FNNNVP

SEQ ID NO: 6 - protein, 9 aa, Synthetic construct
ASSLNTEAF

SEQ ID NO: 7 - protein, 109 aa, Synthetic construct
QQGEEDPQALSIQEGENATMNCSYKTSINNLQWYRQNSGRGLVHLILIRSNEREKHSGRLRVTLDTSKKSSSLLITASRAADTASYFCATDERDDMRFGAGTRLTVKPN

SEQ ID NO: 8 - protein, 109 aa, Synthetic construct
GVIQSPRHEVTEMGQEVTLRCKPISGHDYLFWYRQTMMRGLELLIYFNNNVPIDDSGMPEDRFSAKMPNASFSTLKIQPSEPRDSAVYFCASSLNTEAFFGQGTRLTVV

SEQ ID NO: 9 - protein, 22 aa, Synthetic construct
GSGATNFSLLKQAGDVEENPGP

SEQ ID NO: 10 - protein, 266 aa, Synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDERDDMRFGAGTRLTVKPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 11 - protein, 303 aa, Synthetic construct
MDSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSLNTEAFFGQGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 12 - protein, 591 aa, Synthetic construct
MDSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSLNTEAFFGQGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNSGSGATNFSLLKQAGDVEENPGPMETLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDERDDMRFGAGTRLTVKPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 13 - protein, 5 aa, Synthetic construct
KTLYG

SEQ ID NO: 14 - protein, 7 aa, Synthetic construct
LQKGGEE

SEQ ID NO: 15 - protein, 10 aa, Synthetic construct
GADTSTDKLI

SEQ ID NO: 16 - protein, 5 aa, Synthetic construct
SGHRS

SEQ ID NO: 17 - protein, 6 aa, Synthetic construct
YFSETQ

SEQ ID NO: 18 - protein, 11 aa, Synthetic construct
ASSHGGAYEQY

SEQ ID NO: 19 - protein, 110 aa, Synthetic construct
QELEQSPQSLIVQEGKNLTINCTSSKTLYGLYWYKQKYGEGLIFLMMLQKGGEEKSHEKITAKLDEKKQQSSLHITASQPSHAGIYLCGADTSTDKLIFGTGTRLQVFPN

SEQ ID NO: 20 - protein, 110 aa, Synthetic construct
GVTQTPRYLIKTRGQQVTLSCSPISGHRSVSWYQQTPGQGLQFLFEYFSETQRNKGNFPGRFSGRQFSNSRSEMNVSTLELGDSALYLCASSHGGAYEQYFGPGTRLTVT

SEQ ID NO: 21 - protein, 271 aa, Synthetic construct
METVLQVLLGILGFQAAWVSSQELEQSPQSLIVQEGKNLTINCTSSKTLYGLYWYKQKYGEGLIFLMMLQKGGEEKSHEKITAKLDEKKQQSSLHITASQPSHAGIYLCGADTSTDKLIFGTGTRLQVFPNIQNPDPAVYQLRDSKSSDKSVCLFTDFDSQTNVSQSKDSDVYITDKCVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 22 - protein, 307 aa, Synthetic construct
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSHGGAYEQYFGPGTRLTVTEDLKNVFPPEVAVFEPSKAEIAHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKD

SEQ ID NO: 23 - protein, 603 aa, Synthetic construct
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSHGGAYEQYFGPGTRLTVTEDLKNVFPPEVAVFEPSKAEIAHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRGGSGATNFSLLKQAGDVEENPGPMETVLQVLLGILGFQAAWVSSQELEQSPQSLIVQEGKNLTINCTSSKTLYGLYWYKQKYGEGLIFLMMLQKGGEEKSHEKITAKLDEKKQQSSLHITASQPSHAGIYLCGADTSTDKLIFGTGTRLQVFPNIQNPDPAVYQLRDSKSSDKSVCLFTDFDSQTNVSQSKDSDVYITDKCVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 24 - protein, 10 aa, Synthetic construct
ATDAYGQNFV

SEQ ID NO: 25 - protein, 9 aa, Synthetic construct
ASGSNTEAF

SEQ ID NO: 26 - protein, 110 aa, Synthetic construct
QQGEEDPQALSIQEGENATMNCSYKTSINNLQWYRQNSGRGLVHLILIRSNEREKHSGRLRVTLDTSKKSSSLLITASRAADTASYFCATDAYGQNFVFGPGTRLSVLPY

SEQ ID NO: 27 - protein, 109 aa, Synthetic construct
GVIQSPRHEVTEMGQEVTLRCKPISGHDYLFWYRQTMMRGLELLIYFNNNVPIDDSGMPEDRFSAKMPNASFSTLKIQPSEPRDSAVYFCASGSNTEAFFGQGTRLTVV

SEQ ID NO: 28 - protein, 267 aa, Synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDAYGQNFVFGPGTRLSVLPYIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 29 - protein, 303 aa, Synthetic construct
MDSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASGSNTEAFFGQGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 30 - protein, 592 aa, Synthetic construct
MDSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASGSNTEAFFGQGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNSGSGATNFSLLKQAGDVEENPGPMETLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDAYGQNFVFGPGTRLSVLPYIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 31 - protein, 9 aa, Synthetic construct
FLLTRILTI

SEQ ID NO: 32 - protein, 9 aa, Synthetic construct
FLLTKILTI

SEQ ID NO: 33 - DNA, 327 bp, Synthetic construct
  source 1..327: mol_type = other DNA
cagcagggcgaagaggatccccaggccctgagcattcaggaaggcgagaacgccaccatgaactgcagctacaagaccagcatcaacaacctgcagtggtacagacagaacagcggcagaggcctggtgcacctgatcctgatcagaagcaacgagagagagaagcactccggcagactgagagtgaccctggacaccagcaagaagtccagcagcctgctgatcaccgccagcagagccgccgataccgccagctacttctgcgccaccgacgagcgggacgacatgagatttggcgccggaacccggctgaccgtgaagcccaat

SEQ ID NO: 34 - DNA, 327 bp, Synthetic construct
  source 1..327: mol_type = other DNA
ggcgtgatccagagccccagacacgaagtgaccgagatgggccaggaagtgaccctgcgctgcaagcctatcagcggccacgactacctgttctggtacagacagaccatgatgcggggcctggaactgctgatctacttcaacaacaacgtgcccatcgacgacagcggcatgcccgaggatagattcagcgccaagatgcccaacgccagcttcagcaccctgaagatccagcccagcgagcccagagacagcgccgtgtacttttgcgccagcagcctgaacaccgaggcattctttgggcagggcacccggctgacagtggtg

SEQ ID NO: 35 - DNA, 801 bp, Synthetic construct
  source 1..801: mol_type = other DNA
atggaaacactgctgggagtgtccctcgtgatcctgtggctgcagctggccagagtgaacagccagcagggcgaagaggatccccaggccctgagcattcaggaaggcgagaacgccaccatgaactgcagctacaagaccagcatcaacaacctgcagtggtacagacagaacagcggcagaggcctggtgcacctgatcctgatcagaagcaacgagagagagaagcactccggcagactgagagtgaccctggacaccagcaagaagtccagcagcctgctgatcaccgccagcagagccgccgataccgccagctacttctgcgccaccgacgagcgggacgacatgagatttggcgccggaacccggctgaccgtgaagcccaatatccagaaccccgagcccgccgtgtaccagctgaaggaccccagaagccaggacagcaccctgtgtctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacatgccaggacatcttcaaagagacaaacgccacctacccttccagcgacgtgccctgtgacgccaccctgaccgagaagtccttcgagacagacatgaatctgaatttccagaacctgagcgtgatgggcctgcggatcctgctgctgaaagtggccggcttcaacctgctgatgaccctgcggctgtggtccagctga

SEQ ID NO: 36 - DNA, 909 bp, Synthetic construct
  source 1..909: mol_type = other DNA
atggacagctggaccctgtgttgcgtgtccctgtgcatcctggtggccaagcacacagatgccggcgtgatccagagccccagacacgaagtgaccgagatgggccaggaagtgaccctgcgctgcaagcctatcagcggccacgactacctgttctggtacagacagaccatgatgcggggcctggaactgctgatctacttcaacaacaacgtgcccatcgacgacagcggcatgcccgaggatagattcagcgccaagatgcccaacgccagcttcagcaccctgaagatccagcccagcgagcccagagacagcgccgtgtacttttgcgccagcagcctgaacaccgaggcattctttgggcagggcacccggctgacagtggtggaagatctgaggaacgtgaccccccccaaggtgtccctgttcgagcccagcaaggccgagatcgccaacaagcagaaagccaccctcgtgtgcctggccagaggcttcttccccgaccacgtggaactgtcctggtgggtcaacggcaaagaggtgcacagcggagtctgcaccgacccccaggcttacaaagagagcaactacagctactgcctgtccagcagactgcgggtgtccgctaccttctggcacaacccccggaaccacttcagatgccaggtgcagttccacggcctgagcgaagaggacaagtggcccgagggcagccccaagcccgtgacccagaatatcagcgccgaggcctggggcagagccgattgtggcatcaccagcgccagctaccaccagggggtgctgagcgccaccatcctgtacgagatcctgctgggcaaggccaccctgtacgccgtgctggtgtccggactggtgctgatggccatggtcaagaagaagaacagc

SEQ ID NO: 37 - DNA, 1776 bp, Synthetic construct
  source 1..1776: mol_type = other DNA
atggacagctggaccctgtgttgcgtgtccctgtgcatcctggtggccaagcacacagatgccggcgtgatccagagccccagacacgaagtgaccgagatgggccaggaagtgaccctgcgctgcaagcctatcagcggccacgactacctgttctggtacagacagaccatgatgcggggcctggaactgctgatctacttcaacaacaacgtgcccatcgacgacagcggcatgcccgaggatagattcagcgccaagatgcccaacgccagcttcagcaccctgaagatccagcccagcgagcccagagacagcgccgtgtacttttgcgccagcagcctgaacaccgaggcattctttgggcagggcacccggctgacagtggtggaagatctgaggaacgtgaccccccccaaggtgtccctgttcgagcccagcaaggccgagatcgccaacaagcagaaagccaccctcgtgtgcctggccagaggcttcttccccgaccacgtggaactgtcctggtgggtcaacggcaaagaggtgcacagcggagtctgcaccgacccccaggcttacaaagagagcaactacagctactgcctgtccagcagactgcgggtgtccgctaccttctggcacaacccccggaaccacttcagatgccaggtgcagttccacggcctgagcgaagaggacaagtggcccgagggcagccccaagcccgtgacccagaatatcagcgccgaggcctggggcagagccgattgtggcatcaccagcgccagctaccaccagggggtgctgagcgccaccatcctgtacgagatcctgctgggcaaggccaccctgtacgccgtgctggtgtccggactggtgctgatggccatggtcaagaagaagaacagcggcagcggcgccaccaacttcagcctgctgaagcaggccggcgacgtggaggagaaccccggccccatggaaacactgctgggagtgtccctcgtgatcctgtggctgcagctggccagagtgaacagccagcagggcgaagaggatccccaggccctgagcattcaggaaggcgagaacgccaccatgaactgcagctacaagaccagcatcaacaacctgcagtggtacagacagaacagcggcagaggcctggtgcacctgatcctgatcagaagcaacgagagagagaagcactccggcagactgagagtgaccctggacaccagcaagaagtccagcagcctgctgatcaccgccagcagagccgccgataccgccagctacttctgcgccaccgacgagcgggacgacatgagatttggcgccggaacccggctgaccgtgaagcccaatatccagaaccccgagcccgccgtgtaccagctgaaggaccccagaagccaggacagcaccctgtgtctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacatgccaggacatcttcaaagagacaaacgccacctacccttccagcgacgtgccctgtgacgccaccctgaccgagaagtccttcgagacagacatgaatctgaatttccagaacctgagcgtgatgggcctgcggatcctgctgctgaaagtggccggcttcaacctgctgatgaccctgcggctgtggtccagctga

SEQ ID NO: 38 - DNA, 330 bp, Synthetic construct
  source 1..330: mol_type = other DNA
caagagctggaacagtctccccagagcctgatcgtgcaagagggcaagaacctgaccatcaactgcaccagcagcaagaccctgtacggcctgtactggtacaagcagaagtacggcgagggcctgatcttcctgatgatgctgcagaaaggcggcgaggaaaagagccacgagaagatcaccgccaagctggacgagaagaagcagcagagcagcctgcacatcacagccagccagccttctcacgccggcatctatttgtgcggagccgacaccagcaccgacaagctgatctttggcaccggcacacggctccaggtgttccccaat

SEQ ID NO: 39 - DNA, 330 bp, Synthetic construct
  source 1..330: mol_type = other DNA
ggcgtgacccagacacctagatacctgatcaagaccagaggccagcaagtgaccctgagctgctctcctatcagcggccacagaagcgtgtcctggtatcagcagacacctggacagggcctgcagttcctgttcgagtacttcagcgagacacagcggaacaagggcaacttccccggcagattttccggcagacagttcagcaacagccgcagcgagatgaacgtgtccacactggaactgggcgacagcgccctgtatctgtgtgctagttctcacggcggagcctacgagcagtatttcggccctggcaccagactgaccgtgacc

SEQ ID NO: 40 - DNA, 813 bp, Synthetic construct
  source 1..813: mol_type = other DNA
atggaaaccgtgctgcaggttctgctgggaatcctgggatttcaggccgcctgggtgtccagccaagagctggaacagtctccccagagcctgatcgtgcaagagggcaagaacctgaccatcaactgcaccagcagcaagaccctgtacggcctgtactggtacaagcagaagtacggcgagggcctgatcttcctgatgatgctgcagaaaggcggcgaggaaaagagccacgagaagatcaccgccaagctggacgagaagaagcagcagagcagcctgcacatcacagccagccagccttctcacgccggcatctatttgtgcggagccgacaccagcaccgacaagctgatctttggcaccggcacacggctccaggtgttccccaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgatttcgcctgcgccaacgccttcaacaatagcattatccccgaggacacattcttccccagctccgatgtgccctgcgacgtgaagctggtggaaaagtccttcgagacagacaccaacctgaacttccagaacctgagcgtgatcggcttcagaatcctgctgctgaaggtggccggcttcaacctgctgatgacactgagactgtggtccagc

SEQ ID NO: 41 - DNA, 921 bp, Synthetic construct
  source 1..921: mol_type = other DNA
atgggatctagactgctttgttgggtgctgctgtgcctgcttggagccggacctgtgaaagctggcgtgacccagacacctagatacctgatcaagaccagaggccagcaagtgaccctgagctgctctcctatcagcggccacagaagcgtgtcctggtatcagcagacacctggacagggcctgcagttcctgttcgagtacttcagcgagacacagcggaacaagggcaacttccccggcagattttccggcagacagttcagcaacagccgcagcgagatgaacgtgtccacactggaactgggcgacagcgccctgtatctgtgtgctagttctcacggcggagcctacgagcagtatttcggccctggcaccagactgaccgtgaccgaggatctgaagaacgtgttcccacctgaggtggccgtgttcgagccttctaaggccgagattgcccacacacagaaagccacactcgtgtgtctggccaccggcttctatcccgatcacgtggaactgtcttggtgggtcaacggcaaagaggtgcacagcggcgtctgcacagatccccagcctctgaaagaacagcccgctctgaacgacagccggtactgtctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggaattaccagcgccagctaccatcagggcgtgctgtctgccacaatcctgtacgagatcctgctgggcaaagccactctgtacgccgtgctggtttctgccctggtgctgatggccatggtcaagcggaaggat

SEQ ID NO: 42 - DNA, 1809 bp, Synthetic construct
  source 1..1809: mol_type = other DNA
atgggatctagactgctttgttgggtgctgctgtgcctgcttggagccggacctgtgaaagctggcgtgacccagacacctagatacctgatcaagaccagaggccagcaagtgaccctgagctgctctcctatcagcggccacagaagcgtgtcctggtatcagcagacacctggacagggcctgcagttcctgttcgagtacttcagcgagacacagcggaacaagggcaacttccccggcagattttccggcagacagttcagcaacagccgcagcgagatgaacgtgtccacactggaactgggcgacagcgccctgtatctgtgtgctagttctcacggcggagcctacgagcagtatttcggccctggcaccagactgaccgtgaccgaggatctgaagaacgtgttcccacctgaggtggccgtgttcgagccttctaaggccgagattgcccacacacagaaagccacactcgtgtgtctggccaccggcttctatcccgatcacgtggaactgtcttggtgggtcaacggcaaagaggtgcacagcggcgtctgcacagatccccagcctctgaaagaacagcccgctctgaacgacagccggtactgtctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggaattaccagcgccagctaccatcagggcgtgctgtctgccacaatcctgtacgagatcctgctgggcaaagccactctgtacgccgtgctggtttctgccctggtgctgatggccatggtcaagcggaaggatagcagaggcggaagcggcgccacaaacttcagcctgcttaaacaggccggcgacgtggaagagaaccccggacctatggaaaccgtgctgcaggttctgctgggaatcctgggatttcaggccgcctgggtgtccagccaagagctggaacagtctccccagagcctgatcgtgcaagagggcaagaacctgaccatcaactgcaccagcagcaagaccctgtacggcctgtactggtacaagcagaagtacggcgagggcctgatcttcctgatgatgctgcagaaaggcggcgaggaaaagagccacgagaagatcaccgccaagctggacgagaagaagcagcagagcagcctgcacatcacagccagccagccttctcacgccggcatctatttgtgcggagccgacaccagcaccgacaagctgatctttggcaccggcacacggctccaggtgttccccaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgatttcgcctgcgccaacgccttcaacaatagcattatccccgaggacacattcttccccagctccgatgtgccctgcgacgtgaagctggtggaaaagtccttcgagacagacaccaacctgaacttccagaacctgagcgtgatcggcttcagaatcctgctgctgaaggtggccggcttcaacctgctgatgacactgagactgtggtccagc

SEQ ID NO: 43 - DNA, 330 bp, Synthetic construct
  source 1..330: mol_type = other DNA
cagcagggcgaagaggatccccaggccctgagcattcaggaaggcgagaacgccaccatgaactgcagctacaagaccagcatcaacaacctgcagtggtacagacagaacagcggcagaggcctggtgcacctgatcctgatcagaagcaacgagagagagaagcactccggcagactgagagtgaccctggacaccagcaagaagtccagcagcctgctgatcaccgccagcagagccgccgataccgccagctacttctgtgccacagacgcctacggccagaacttcgtgtttggccctggcaccagactgagcgtgctgccctat

SEQ ID NO: 44 - DNA, 327 bp, Synthetic construct
  source 1..327: mol_type = other DNA
ggcgtgatccagagccccagacacgaagtgaccgagatgggccaggaagtgaccctgcgctgcaagcctatcagcggccacgactacctgttctggtacagacagaccatgatgcggggcctggaactgctgatctacttcaacaacaacgtgcccatcgacgacagcggcatgcccgaggatagattcagcgccaagatgcccaacgccagcttcagcaccctgaagatccagcccagcgagcccagagacagcgccgtgtacttttgtgccagcggcagcaacaccgaggcattctttgggcagggcacccggctgacagtggtg

SEQ ID NO: 45 - DNA, 804 bp, Synthetic construct
  source 1..804: mol_type = other DNA
atggaaacactgctgggagtgtccctcgtgatcctgtggctgcagctggccagagtgaacagccagcagggcgaagaggatccccaggccctgagcattcaggaaggcgagaacgccaccatgaactgcagctacaagaccagcatcaacaacctgcagtggtacagacagaacagcggcagaggcctggtgcacctgatcctgatcagaagcaacgagagagagaagcactccggcagactgagagtgaccctggacaccagcaagaagtccagcagcctgctgatcaccgccagcagagccgccgataccgccagctacttctgtgccacagacgcctacggccagaacttcgtgtttggccctggcaccagactgagcgtgctgccctatatccagaaccccgagcccgccgtgtaccagctgaaggaccccagaagccaggacagcaccctgtgtctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacatgccaggacatcttcaaagagacaaacgccacctacccttccagcgacgtgccctgtgacgccaccctgaccgagaagtccttcgagacagacatgaatctgaatttccagaacctgagcgtgatgggcctgcggatcctgctgctgaaagtggccggcttcaacctgctgatgaccctgcggctgtggtccagctga

SEQ ID NO: 46 - DNA, 909 bp, Synthetic construct
  source 1..909: mol_type = other DNA
atggacagctggaccctgtgttgcgtgtccctgtgcatcctggtggccaagcacacagatgccggcgtgatccagagccccagacacgaagtgaccgagatgggccaggaagtgaccctgcgctgcaagcctatcagcggccacgactacctgttctggtacagacagaccatgatgcggggcctggaactgctgatctacttcaacaacaacgtgcccatcgacgacagcggcatgcccgaggatagattcagcgccaagatgcccaacgccagcttcagcaccctgaagatccagcccagcgagcccagagacagcgccgtgtacttttgtgccagcggcagcaacaccgaggcattctttgggcagggcacccggctgacagtggtggaagatctgaggaacgtgaccccccccaaggtgtccctgttcgagcccagcaaggccgagatcgccaacaagcagaaagccaccctcgtgtgcctggccagaggcttcttccccgaccacgtggaactgtcctggtgggtcaacggcaaagaggtgcacagcggagtctgcaccgacccccaggcttacaaagagagcaactacagctactgcctgtccagcagactgcgggtgtccgctaccttctggcacaacccccggaaccacttcagatgccaggtgcagttccacggcctgagcgaagaggacaagtggcccgagggcagccccaagcccgtgacccagaatatcagcgccgaggcctggggcagagccgattgtggcatcaccagcgccagctaccaccagggggtgctgagcgccaccatcctgtacgagatcctgctgggcaaggccaccctgtacgccgtgctggtgtccggactggtgctgatggccatggtcaagaagaagaacagc

SEQ ID NO: 47 - DNA, 1779 bp, Synthetic construct
  source 1..1779: mol_type = other DNA
atggacagctggaccctgtgttgcgtgtccctgtgcatcctggtggccaagcacacagatgccggcgtgatccagagccccagacacgaagtgaccgagatgggccaggaagtgaccctgcgctgcaagcctatcagcggccacgactacctgttctggtacagacagaccatgatgcggggcctggaactgctgatctacttcaacaacaacgtgcccatcgacgacagcggcatgcccgaggatagattcagcgccaagatgcccaacgccagcttcagcaccctgaagatccagcccagcgagcccagagacagcgccgtgtacttttgtgccagcggcagcaacaccgaggcattctttgggcagggcacccggctgacagtggtggaagatctgaggaacgtgaccccccccaaggtgtccctgttcgagcccagcaaggccgagatcgccaacaagcagaaagccaccctcgtgtgcctggccagaggcttcttccccgaccacgtggaactgtcctggtgggtcaacggcaaagaggtgcacagcggagtctgcaccgacccccaggcttacaaagagagcaactacagctactgcctgtccagcagactgcgggtgtccgctaccttctggcacaacccccggaaccacttcagatgccaggtgcagttccacggcctgagcgaagaggacaagtggcccgagggcagccccaagcccgtgacccagaatatcagcgccgaggcctggggcagagccgattgtggcatcaccagcgccagctaccaccagggggtgctgagcgccaccatcctgtacgagatcctgctgggcaaggccaccctgtacgccgtgctggtgtccggactggtgctgatggccatggtcaagaagaagaacagcggcagcggcgccaccaacttcagcctgctgaagcaggccggcgacgtggaggagaaccccggccccatggaaacactgctgggagtgtccctcgtgatcctgtggctgcagctggccagagtgaacagccagcagggcgaagaggatccccaggccctgagcattcaggaaggcgagaacgccaccatgaactgcagctacaagaccagcatcaacaacctgcagtggtacagacagaacagcggcagaggcctggtgcacctgatcctgatcagaagcaacgagagagagaagcactccggcagactgagagtgaccctggacaccagcaagaagtccagcagcctgctgatcaccgccagcagagccgccgataccgccagctacttctgtgccacagacgcctacggccagaacttcgtgtttggccctggcaccagactgagcgtgctgccctatatccagaaccccgagcccgccgtgtaccagctgaaggaccccagaagccaggacagcaccctgtgtctgttcaccgacttcgacagccagatcaacgtgcccaagaccatggaaagcggcaccttcatcaccgataagtgcgtgctggacatgaaggccatggacagcaagagcaacggcgccattgcctggtccaaccagaccagcttcacatgccaggacatcttcaaagagacaaacgccacctacccttccagcgacgtgccctgtgacgccaccctgaccgagaagtccttcgagacagacatgaatctgaatttccagaacctgagcgtgatgggcctgcggatcctgctgctgaaagtggccggcttcaacctgctgatgaccctgcggctgtggtccagctga